(19) United States
(12) Patent Application Publication
(10) Pub. No.: US 2008/0114096 A1
(43) Pub. Date: May 15, 2008

(54) Lubricious biopolymeric network compositions and methods of making same

(75) Inventors: Xin Qu, Hefei (CN); Tom Schottman, Flemington, NJ (US); Rainer Gruening, Basking Ridge, NJ (US); Paul N. Chen, Canandaigua, NY (US); Joseph Conte, Monroe Twsp., NJ (US); Zheng Wang, Bridgewater, NJ (US); Paaquale P. Vicario, North Plainfield, NJ (US); Zichun Lu, Newton, PA (US); Karen Meritt, Avon-By-The-Sea, NJ (US); Dave Buongiovanni, Manville, NJ (US)

Correspondence Address: HOFFMANN & BARON, LLP, 6900 JERICHO TURNPIKE, SYOSSET, NY, 11791, US

(73) Assignee: Hydromer, Inc.

(21) Appl. No.: 11/598,223

(22) Filed: Nov. 9, 2006

(51) Int. Cl. A61K 8/73; C08L 1/00

(52) U.S. Cl. 524/18; 427/294; 427/427.4; 427/457; 524/27; 524/35; 524/47; 524/56; 524/58

(57) ABSTRACT

The invention provides a network composition comprising a plurality of associated saccharide chains wherein a chain comprises at least one saccharide component; and at least one first monomer, at least one second monomer, or combinations of both; wherein the first monomer is linked to the saccharide component by ester linkages; ether linkages; amide linkages; ketone linkages; or combinations thereof; wherein the second monomer is linked to the saccharide component by ester linkages; ether linkages; amide linkages; ketone linkages; urea linkages; carbamate linkages, aluminum oxide linkages; siloxane linkages; or combinations thereof; wherein first monomers are linked to each other by ester linkages; ether linkages; amide linkages; ketone linkages; or combinations thereof; wherein the first monomer is linked to the second monomer by ester linkages; ether linkages; amide linkages; ketone linkages; urea linkages; carbamate linkages; aluminum oxide linkages; siloxane linkages or combinations thereof; wherein the second monomers are linked to each other by ester linkages; ether linkages; amide linkages; ketone linkages; polyvinyl linkages, polyolefin linkages, urea linkages; carbamate linkages, aluminum oxide linkages, siloxane linkages or combinations thereof; and wherein the saccharide components are linked to each other by ester linkages; ether linkages; amide linkages; ketone linkages; or combinations thereof; wherein the network is capable of adhering to a substrate.

### TECHNICAL FIELD

The present invention relates to biocompatible, highly lubricious, durable coating compositions and methods for coating the surface of medical devices with a natural based film forming polymeric material.

### BACKGROUND OF THE INVENTION

Advances in the design of medical devices, such as catheters, guide wires and stents, have greatly improved the quality of medical care. However, these devices are often made with materials that cause undesirable complications, such as bacterial infection, blood clots, inflammation and tissue trauma caused by device insertion. Coatings are placed on the medical device in an attempt to alleviate these challenges without altering the device's bulk material properties.

For example, hydrophilic coatings with low friction (coefficient of friction of 0.3 or less) are useful for a variety of medical devices such as catheters, catheter introducers and the like. Upon introduction into the body, these coated devices slide easily within arteries, veins and other body orifices and passageways. Examples of such coating materials include coatings based on polyvinylpyrrolidone, poly(ethylene oxide) and polyurethane as described in U.S. Pat. Nos. 4,642,267 and 6,461,311. However, these coating materials typically lack other desirable properties, such as biocompatiblity.

Accordingly, therapeutic agents have been included into coating materials. In particular, some medical device coatings are made to release pharmaceutically-active agents via dissolution of the active or by cleavage of the active from the coating.

For example, U.S. Pat. No. 5,163,958 discloses a stent having a binder layer and an anti-thrombogenic pyrolytic amorphous carbon layer attached to the binder layer to provide an anti-thrombogenic surface. Also, U.S. Pat. No. 5,342,348 discloses porous polyurethane and PTFE stents having biodegradable polymeric filaments which release a drug over time. Additionally, polymeric films for slow or controlled release of an active agent into the body have been suggested, for example, U.S. Pat. No. 5,383,928 discloses delivery of a drug using a stent-sheath structure made from both degradable and non-degradable polymers, such as ethylene vinyl acetate (EVA).

However, these prior art coatings have substantial disadvantages. For instance, these coatings may be cytotoxic. Additionally, due to the localized drug elution, such coatings and can lead to hemolysis, or to the formation of emboli or clots in blood vessels. Other adverse reactions may include inflammation and cell proliferation which can lead to hyperplasia, occlusion of blood vessels, platelet aggregation, rejection of artificial organs, and calcification.

Further drawbacks of conventional biocompatible coatings are the use of substantial amounts of organic solvents and/or UV curing in the process of making the coatings. These drawbacks complicate the process of making the coatings. Also, the organic solvents may be highly reactive in vivo if they are not completely removed prior to implantation.

Additionally, polyurethane coatings may suffer from stability problems. For example, such coatings may have limited substrate adhesion, may abrade easily during multiple insertion of the coated device, may cause blood coagulation, may cause cell-biological adverse effects such as increased cell mytosis or may cause inflammation, are quickly bio-degraded and/or bio-eroded.

Accordingly, there is a critical need in the medical field for medical device coatings that simultaneously exhibit a low coefficient of friction, are biocompatible, are durable and are readily processed.

### SUMMARY OF THE INVENTION

The present invention relates to biocompatible, highly lubricious, durable coating compositions and methods for coating the surfaces of medical devices with a natural based film forming polymeric material.

In one embodiment, the compositions are non-leachable. In this embodiment, the network composition is formed by contacting: a multifunctional monomer component consisting of a first monomer and a second monomer, and a saccharide component. In another embodiment, the compositions exhibit controlled leaching. In this embodiment, there is only trace amounts of the second monomer, or an absence of the second monomer.

The first monomer has one set of functional groups selected from the list consisting of hydroxyl/aldehyde; aldehyde/carboxylic acid; hydroxyl/carboxylic acid; aldehyde/amine; carboxylic acid/amine; amine/amine; carboxylic acid/carboxylic acid; hydroxyl/amine; hydroxyl/hydroxyl; aldehyde/aldehyde and combinations thereof.

The second monomer has at least one set of functional group selected from the list consisting of hydroxyl/carboxylic acid; carboxylic acid/carboxylic acid; hydroxyl/aldehyde; aldehyde/carboxylic acid; aldehyde/aldehyde; hydroxyl/hydroxyl; carboxylic acid/vinyl; amine/carboxylic acid; amine/amine; hydroxyl/vinyl/carboxylic acid; hydroxyl/olefin/carboxylic acid; olefin/carboxylic acid; carboxylic anhydride; carboxylic acid anhydride/hydroxyl; carboxylic acid anhydride/aldehyde; carboxylic acid anhydride/olefin; carboxylic acid anhydride/vinyl; carboxylic acid anhydride/amine; hydroxyl/olefin; hydroxyl/amine; aldehyde/olefin; aldehyde/vinyl; aldehyde/amine; aziridine; aziridine derivatives; epoxides; blocked isocyanates; colloidal silica; colloidal alumina; and combinations thereof.

The ratio of the first monomer to the second monomer is from about 5:1 to about 50:1 for the compositions that are non-leachable. The second monomer is absent or virtually absent in the compositions that exhibit controlled leaching.

The saccharide component contains functional groups which can associate with the first and second monomers, wherein the weight ratio of the saccharide component to the monomer component ranges from about 1:50 to about 10:1.

The monomer component and the saccharide component are contacted in the presence of a solvent composition. Upon evaporation of the solvent composition, the network composition forms and a film of the composition adheres to a substrate. The network composition reduces the coefficient of friction of the substrate by at least about 85%. Typically, the substrate is a medical device.

In other embodiments, the ratio of the first monomer to the second monomer is from about 20:1 to about 30:1. The weight ratio of the saccharide component to the monomer component is from about 1:10 to about 2:1.

The first monomer and the second monomer each individually comprise from about 2 to about 24 carbon atoms.

The saccharide component comprises polysaccharides, oligosaccharides, trisaccharides, disaccharides, monosaccharides, or derivatives thereof or combinations thereof. In particular, the saccharide component comprises polyols, cellulosics, chitosans, heparin, starch, sugar, homopolysaccharides, heteropolysaccharides, glucosamines, or derivatives thereof, or combinations thereof. The chitosans and chitosan derivatives are chosen from the group consisting of chitin, deacetylated chitin, N-carboxymethyl chitosan, O-carboxymethyl chitosan, N,O-carboxymethyl chitosan, carboxypropyl chitosan, carboxybutyl chitosan, hydrolized chitosan, chitosan adipate, chitosan ascorbate, chitosan formate, chitosan glycolate, polyquaternium-29, chitosan PCA (pyrrolidone carboxylic acid salt of chitosan), myristoyl/PCA chitin, chitosan lactate, chitosan lauroyl glycinate, chitosan salicylate, chitosan succinimide, galactosylated chitosan, hydroxyethyl chitosan, hydroxypropyl chitosan, and amino derivatives thereof, aldehyde derivatives thereof, carboxylic acid derivatives thereof, and combinations thereof. In a preferred embodiment, the saccharide component comprises heparin, or derivatives thereof, or combinations thereof.

Examples of cellulosics are cellulose, polyquaternium-4, polyquaternium-10, polyquaternium-4/hydroxypropyl starch copolymer, polyquaternium-24, cellulose acetate, cellulose acetate butyrate, cellulose acetate propionate, cellulose acetate propinate carboxylate, cellulose gum, cellulose succinate, carboxy cellulose, aminocellulose, aminocellulose tosylates, and amino derivatives thereof, aldehyde derivatives thereof, carboxylic acid derivatives thereof, and combinations thereof.

The polysaccharides and polysaccharide derivatives are chosen from the group consisting of corn starch, hydrolyzed wheat protein, hydrolyzed wheat protein/PVP crosspolymer, glycogen, gelatin, inulin, pectin, heparin salts, hyaluronan, carregannan, algennan, algenic acid, alginate, gum arabic, locust bean gum, agar-agar, carrageenans, guar gum, xanthan gum, aloe barbadesis polysaccharides, arbutin, glucosic acid, glucodides, their amino derivatives, aldehyde derivatives, carboxylic acid derivatives and combinations thereof.

The sugar is glucose, fructose, mannose, galactose, algae oligosaccharides, their amino derivatives, aldehyde derivatives, carboxylic acid derivatives, d-(alpha or beta)glycosamine, d-(alpha or beta)galactoseamine and alkyl derivatives of these amino sugars.

The saccharide component comprises functional groups selected from the list consisting of hydroxyl; aldehyde; carboxylic acid; carboxyalkyl acid; amine; alkyl-amine; vinyl saccharides, saccharides containing olefinic side chains, saccharide isocyanates, —SH, —S-Alkyl, —SO4-, —SO3-, sulfonamides, SNH-alkyl; and combinations thereof.

Examples of the first monomer are an alcohol; an aldehyde; an glutaraldehyde; lactic acid; salicylic acid; p-hydroxybenzoic acid; citric acid; glycerin acid; alanine; glutamic acid; a primary amine; a carboxylic acid; dicarboxylic acid anhydride; a hydroxydicarboxylic acid; an alpha-amino acid; a beta-amino acid; a gamma-amino acid; an omega-amino acid; an alpha-hydroxy carboxylic acid; a beta-hydroxy carboxylic acid; a gamma-hydroxy carboxylic acid, an omega-hydroxy carboxylic acid; an alpha-hydroxy aldehyde; a beta-hydroxy aldehyde; a gamma-hydroxy aldehyde; an omega-hydroxy aldehyde; an alpha-aldehyde carboxylic acid; a beta-aldehyde carboxylic acid; a gamma-aldehyde carboxylic acid; an omega-aldehyde carboxylic acid; a diamine; and a hydroxy amine.

Examples of the second monomer are acrylic acid, an alcohol; an aldehyde; an glutaraldehyde; aspartamic acid; aspartame; lactic acid; salicylic acid; p-hydroxybenzoic acid; maleic acid; citric acid; sorbic acid; glycerin acid; alanine; glutamic acid; a primary amine; a carboxylic acid; a dicarboxylic acid anhydride; a hydroxydicarboxylic acid; an alpha-amino acid; a beta-amino acid; a gamma-amino acid; an omega-amino acid; an alpha-hydroxy carboxylic acid; a beta-hydroxy carboxylic acid; a gamma-hydroxy carboxylic acid, an omega-hydroxy carboxylic acid; an alpha-hydroxy aldehyde; a beta-hydroxy aldehyde; a gamma-hydroxy aldehyde; an omega-hydroxy aldehyde; an alpha-aldehyde carboxylic acid; a beta-aldehyde carboxylic acid; a gamma-aldehyde carboxylic acid; an omega-aldehyde carboxylic acid; a diamine; a hydroxy amine; an alpha-olefinic carboxylic acid; a beta-olefinic carboxylic acid; a gamma-olefinic carboxylic acid; an omega olefinic carboxylic acid; an alkylated acrylic acid; a hydroxyalkylated acrylic acid; an amino acrylic acid; an aminoalkylated acrylic acid; an alpha dimethylacrylic acid; a beta dimethylacrylic acid; a hydroxyacrylic acid, a semialdehyde; ginipin; hydroxyethylmethylacrylate (HEMA); hydroxypropyl methylacrylate (HPMA); colloidal silica; colloidal alumina; an epoxide; a melamine, an aziridine; a carbodiimide; a blocked di-isocyanate; a blocked multiisocyanate; a blocked di-thioisocyanate; and a blocked multithioisocyanate.

The solvent composition comprises water, alcohols, alkylketones, arylalkylketones, ketoalcohols, cyclic ketones, heterocyclic ketones, ethers, cyclic ethers, esters and combinations thereof. Examples of the solvent composition comprises methanol, ethanol, propanol, isopropanol, butanol, methyl ethyl ketone, tetrahydrofuran, acetone, diacetone alcohol, N-methylpyrrolidone, dimethylsulfoxide, (DMSO), dimethylformamide (DMF) and combinations thereof.

In some embodiments, the network composition further comprise a film-improving ingredient, a biologically active material or a combination of both. Examples of the film-improving ingredient is selected from the group consisting of a surfactant, a wetting agent, a plasticizer, a humectant, a viscosity modifier, a defoamer, an emulsifier, a dye, a pigment, a colorant, a UV absorber, a radical scavenger, an antioxidant, an anti-corrosion agent, a carbon dioxide releaser, an optical brightener, a fluorescer, a bleach, a bleach activator, a bleach catalyst, a non-activated enzyme, an enzyme stabilizing system, a chelant, a coating aid, a metal catalyst, a metal oxide catalyst, an organometallic catalyst, a film forming promoter, a hardener, a linking accelerator, a flow agent, a leveling agent, a lubricant, a matte particle, a rheological modifier, a thickener, an electrolyte, a conductive or non-conductive metal oxide particle, a colloidal antimicrobial metal oxide, a magnetic particle, an anti-static agent, a pH control agent, a perfume, a preservative, a biocide, a pesticide, an anti-fouling agent, an algicide, a bactericide, a germicide, a disinfectant, a fungicide, a bio-effecting agent, a vitamin or combinations thereof.

Examples of the biologically active material is an antithrombotic agent, a biostatic agent, a cytostatic agent, a radiation emitter, a pharmaceutical, a biomolecule, an anti-inflammatory agent, an immunosuppressant, an antibiotic, an antiseptic, or combinations thereof. The biologically active material is linked by chemical functional group interaction to the composition or is physically embedded in the composition.

In another aspect of the invention, a method of making a network composition is provided. The method comprises: contacting a multifunctional monomer component and a saccharide component in the presence of a solvent composition to form an reaction solution, and evaporating the solvent composition to form the network composition, wherein the multifunctional monomer component comprises a first monomer and a second monomer, wherein the first monomer has one set of functional groups selected from the list consisting of hydroxyl/aldehyde; aldehyde/carboxylic acid; hydroxyl/carboxylic acid; aldehyde/amine; carboxylic acid/amine; amine/amine; carboxylic acid/carboxylic acid; hydroxyl/amine; hydroxyl/hydroxyl; aldehyde/aldehyde and combinations thereof, and wherein the second monomer has at least one set of functional group selected from the list consisting of hydroxyl/carboxylic acid; carboxylic acid/carboxylic acid; hydroxyl/aldehyde; aldehyde/carboxylic acid; aldehyde/aldehyde; hydroxyl/hydroxyl; carboxylic acid/vinyl; amine/carboxylic acid; amine/amine; hydroxyl/vinyl/carboxylic acid; hydroxyl/olefin/carboxylic acid; olefin/carboxylic acid; carboxylic anhydride; carboxylic acid anhydride/hydroxyl; carboxylic acid anhydride/aldehyde; carboxylic acid anhydride/olefin; carboxylic acid anhydride/vinyl; carboxylic acid anhydride/amine; hydroxyl/olefin; hydroxyl/amine; aldehyde/olefin; aldehyde/vinyl; aldehyde/amine; aziridine; aziridine derivatives; epoxides; blocked isocyanates; colloidal silica; colloidal alumina; and combinations thereof,

The ratio of the first monomer to the second monomer is from about 5:1 to about 100:1, or wherein the second monomer is absent. The saccharide component contains functional groups which can associate with the first and second monomers, wherein the weight ratio of the saccharide component to the monomer component ranges from about 1:50 to about 10:1.

The saccharide component makes up about 0.01 wt. % to about 20 wt. % of the reaction solution. Alternatively, the saccharide component makes up about 0.1 wt. % to about 10 wt. % of the reaction solution. The multifunctional monomer component makes up about 0.001 wt. % to about 30 wt. % of the reaction solution. Alternatively, the multifunctional monomer component makes up about 0.01 wt. % to about 15 wt. % of the reaction solution. The solvent composition makes up about 99.99% wt. to about 50% wt. % of the reaction solution.

In another aspect of the invention, a network composition comprising a plurality of associated saccharide chains is provided. A chain comprises at least one saccharide component; and at least one first monomer, at least one second monomer, or combinations of both. The first monomer is linked to the saccharide component by ester linkages; ether linkages; amide linkages; ketone linkages; or combinations thereof. The second monomer is linked to the saccharide component by ester linkages; ether linkages; amide linkages; ketone linkages; urea linkages; carbamate linkages, aluminum oxide linkages; siloxane linkages; or combinations thereof. First monomers are linked to each other by ester linkages; ether linkages; amide linkages; ketone linkages; or combinations thereof. The first monomer is linked to the second monomer by ester linkages; ether linkages; amide linkages; ketone linkages; urea linkages; carbamate linkages; aluminum oxide linkages; siloxane linkages or combinations thereof. The second monomers are linked to each other by ester linkages; ether linkages; amide linkages; ketone linkages; polyvinyl linkages, polyolefin linkages, urea linkages; carbamate linkages, aluminum oxide linkages, siloxane linkages or combinations thereof. The saccharide components are linked to each other by ester linkages; ether linkages; amide linkages; ketone linkages; or combinations thereof. Preferably, the saccharide component comprises heparin, or derivatives thereof, or combinations thereof.

In another aspect of the invention, an object which comprises the network compositions are provided. The object is preferably made of metal, stainless steel, Nitinol®, plastics, polymers, glass, ceramics, cellulose fibers, synthetic fibers, textiles and alike. Examples of the object include non-expendable or expendable stent, catheter, guide wire, shunt, screw, pin, prosthesis, plate, film, sponge, suture, medical tubing, cannula, balloon, needle, marker, stylet, surgical rod, guidewire tubes, coiled guiding tubes, coiled catheters, electrodal coils, blades, fibers, wound dressing fibers, band aids, suture threads, ocular lens delivery device, ocular lenses, ocular catheters, dialysis catheters, wound drain, or bone implants.

Preferably, the network composition is applied to the object by dipping, spraying, flooding, foaming, roll-coating, brushing, electrolytic depositing, electrostatic spraying, electroplating, vacuum treatment, pressure treatment or combinations thereof.

The network coatings of the present invention provide several advantages over prior art coatings. For example, these coatings are simultaneously biocompatible, highly lubricious, and durable. Additionally, these coatings intrinsically have anti-coagulant and anti-microbial properties. Thus, unlike prior art coatings, the coatings of the present invention do not require the additional of an eluding drug for such therapeutic effects. The fact that eluding drugs are not necessary is highly beneficial. For example, the risk of hemolysis or the formation of emboli by localized drug elution is greatly reduced. Additionally, coatings that require eluding actives for antimicrobial properties lose their activity within days; whereas, the coatings of the present invention retain their antimicrobial activity for extended periods of time.

A further advantage of the present invention coatings is that they are readily processed. For example, unlike prior art coatings, the coatings of the present invention require only limited use of organic solvents. Additionally, the process of making the present invention coatings does not require the pretreatment of substrates, such as plasma or corona treatment, and does not require UV curing. Accordingly, a simplistic manner by which to make the innovative coatings is provided.

### DETAILED DESCRIPTION OF THE INVENTION

The present invention provides biopolymeric network compositions. These compositions exhibit novel cell-biological properties, such as significantly preventing microbial colonization, preventing blood coagulation over extended periods of time, having no hemolytic side effects, having no cytotoxicity, and retarding cell proliferation in a desired controlled way. The compositions exhibit these properties without addition of any drugs. Additionally, the compositions are suitable as lubricious coatings or films, and adhere to polymeric and metallic substrates commonly used for the design of medical devices. The compositions can be designed to be non-leaching or to exhibit a controlled leaching.

Throughout this specification, there are ranges defined by upper and lower boundaries. Each lower boundary can be combined with each upper boundary to define a range. The lower and upper boundaries should each be taken as a separate element.

The biopolymeric network compositions are based on saccharides. The compositions can be formed by any means that forms the complex saccharide network of the present invention. In a preferred embodiment, the compositions are formed by contacting a multifunctional monomer component and a saccharide component in the presence of a solvent composition. Upon removal of the solvent composition, the multifunctional monomer component and the saccharide component link together to form the complex saccharide network.

The multifunctional monomer component, saccharide component and solvent composition are contacted with each other in any order that would allow the formation of the network. Preferably, the multifunctional monomer component, saccharide component and solvent composition are contacted with each other simultaneously

“Saccharide Component”

For the purposes of this specification, a “saccharide component” is defined as any polysaccharide, oligosaccharide, sugar, or derivatives thereof or combinations thereof. For example, the “saccharide component” can be polyols, cellulosics, chitosans, heparin, starch, homopolysaccharides, heteropolysaccharides, glucosamines, or derivatives thereof, or combinations thereof. Saccharide components which are suitable for use in the present invention are natural, semi-natural or synthetic, and are branched, straight-chained, or a mixture of both.

The molecular weight of a saccharide component can range from about 100 g/mol to about 5,000,000 g/mol. For chitosan, the molecular weight can range from about 1000 g/mol up to about 2,000,000 g/mol.

Examples of chitosans and chitosan derivatives which are suitable for the present invention include chitin, deacetylated chitin, N-carboxymethyl chitosan, O-carboxymethyl chitosan, N,O-carboxymethyl chitosan, carboxypropyl chitosan, carboxybutyl chitosan, hydrolized chitosan, chitosan adipate, chitosan ascorbate, chitosan formate, chitosan glycolate, polyquaternium-29, chitosan PCA (pyrrolidone carboxylic acid salt of chitosan), myristoyl/PCA chitin, chitosan lactate, chitosan lauroyl glycinate, chitosan salicylate and chitosan succinimide, galactosylated chitosan, hydroxyethyl chitosan, hydroxypropyl chitosan, and amino derivatives thereof, aldehyde derivatives thereof, carboxylic acid derivatives thereof, and combinations thereof.

Chitin is an unbranched linear polysaccharide of N-acetyl-D-glucosamine units linked by β-1,4 bonds. It is a polymer of glucose in which the hydroxyl group on C-2 is replaced by the N-acetylamino group —NHCOCH₃. In chitosan, the acetyl group is absent. Therefore, chitosan is a deacetylated chitin. Chitosan contains approximately 7% nitrogen and is structurally similar to cellulose. Chitin occurs in nature in the exoskeletons of arthropods such as crabs, lobsters and shrimp. Chitin can be obtained from these sources as an amorphous powder after dissolution of the calcium carbonate with mineral acids and removal of the proteins. It is also found in some fungi, algae and yeast.

Chitosan derivatives are commercially available as, for example, chitosan neutralized with pyrrolidone carboxylic acid available as Kytamer PCA from Amerchol Corporation; carboxymethyl sodium salt of chitosan available as Chitisol from Muto Corporation; chitosan neutralized with glutamic acid available as Seacure+210 from Protan Corporation; N,O-carboxymethyl chitosan available from Nova Chem Ltd., Canada; and un-neutralized chitosan available from Tokyo Kasei Inc.

Plant originated and purified cellulosics are suitable for the present invention including, for example, cellulose, polyquaternium-4, polyquaternium-10, polyquaternium-4/hydroxypropyl starch copolymer, polyquaternium-24, cellulose acetate, cellulose acetate butyrate, cellulose acetate propionate, cellulose acetate propinate carboxylate, cellulose gum, cellulose succinate, carboxymethyl cellulose, aminocellulose, aminocellulose tosylates, and amino derivatives thereof, aldehyde derivatives thereof, carboxylic acid derivatives thereof, and combinations thereof.

Further examples of polysaccharides, oligosaccharides and derivatives thereof which are suitable in the present invention include corn starch and starch of other plants, hydrolyzed wheat protein, hydrolyzed wheat protein/PVP crosspolymer, glycogen, gelatin, inulin, pectin, algennan, algenic acid, alginate, gum arabic, locust bean gum, agar-agar, carrageenans, guar gum, xanthan gum, aloe barbadesis polysaccharides, arbutin, glucosic acid, glucodides, their amino derivatives, aldehyde derivatives, carboxylic acid derivatives and combinations thereof. The saccharide component can also be algae, their amino derivatives, aldehyde derivatives, carboxylic acid derivatives and combinations thereof.

Sugars are cyclic polyols (six and five membered rings), such as monosaccharides, disaccharides, and trisaccharides, including glucose, fructose, mannose, hexose, galactose and their amino derivatives, aldehyde derivatives, carboxylic acid derivatives and combinations thereof. A preferred example of a sugar is d-(alpha or beta)glycosamine, d-(alpha or beta)galactoseamine and alkyl derivatives of these amino sugars.

Besides chitosans, other examples of animal originated polysaccachides and polysaccharide derivatives suitable as the saccharide component include heparin, heparin salts, polysaccharide-based heparin substitutes, hyaluronan, collagen, and mucopolysaccharides found in, for example, green lipped mussel powder and shark cartilage powder. Natural substances of mucopolysaccharides include glucosamine glycans, glucosamine sulphate, chondroitin sulphate and amino sugar.

Before being combined with the multifunctional component, the saccharide component comprises functional groups that associate with the multifunctional monomer component. The functional groups on the saccharide component are selected from the list consisting of hydroxyl; aldehyde; carboxylic acid; carboxyalkyl acid; amine; alkyl-amine; vinyl saccharides, saccharides containing olefinic side chains, saccharide isocyanates, —SH, —S-Alkyl, —SO4-, —SO3-, sulfonamides, SNH-alkyl; and combinations thereof.

Functional groups are naturally present on a saccharide component, or can be added to a saccharide component by derivatization reactions. Examples of typical methods of derivatization include oxidation, carboxylation, amination, sulfonation, phosgenation, dehydrogenation and vinylation. Other methods of derivatization include controlled biological processes, such as bioconversion e.g., enzymatic modification and fermentation. Naturally-occurring functional groups can be modified or simply just purified by modern selective separation methods. For example, the naturally-occurring functional groups can be utilized as is, or functional groups containing links can be cleaved to make functional groups available.

For example, derivatized chitin can be obtained by processes known in the art by which specific chitin chains are deacetylated thereby converting chains partially into a deacetylated state, commonly represented by the % deacetylation usually ranging from 30 to 98%. The deacetylation process make available free primary amine groups which constitute active sites suitable for the present invention. The preferred range of % deacetylation is from about 50% to about 95%.

In some embodiments, the saccharide component is synthesized based on natural saccharide monomer chains which contain certain fractions of derivatized saccharide monomers in their chains. The monomeric aminoglucose, amino mannose, aminoribose, sulfonated saccharides, and glucose sulfates are a few representative examples used in processes called “glycosylation.”

In a preferred embodiment, the saccharide component consists essentially of heparin, and/or derivatives and combinations thereof.

“Multifunctional Monomer Component”

For the non-leaching network compositions, the multifunctional monomer component comprises, or consists essentially of, a first monomer and a second monomer. For the controlled-leaching network compositions, the second monomer is absent, or only present at a low level. First and second monomers that are suitable to interact with the saccharide component are monomeric organic chemical compounds with at least two equal or mixed functional groups.

The first monomer and the second monomer each individually comprise from about 2 to about 24 carbon atoms, more preferably from about 3 to about 20 carbon atoms, and most preferably from about 4 to about 10 carbon atoms.

The first monomer preferably has up to two functional groups, that is, the first monomer is a dual functional monomer. Two functional groups that occur together on a monomer are herein termed as “a set of functional groups.” The first monomer has one set of functional groups selected from the list consisting of: hydroxyl/aldehyde; aldehyde/carboxylic acid; hydroxyl/carboxylic acid; aldehyde/amine; carboxylic acid/amine; amine/amine; carboxylic acid/carboxylic acid; hydroxyl/amine; hydroxyl/hydroxyl; aldehyde/aldehyde and combinations thereof.

An example of a first monomer with the carboxylic acid/amine set is alanine (CH3-CHNH2-COOH). An example of a first monomer with the hydroxy/carboxylic acid set is lactic acid (CH3-CHOH—COOH).

Further examples of a first monomer include alcohols; aldehydes; glutaraldehyde; lactic acid; salicylic acid; p-hydroxybenzoic acid; citric acid; glycerin acid; alanine; glutamic acid; primary amines; carboxylic acids; dicarboxylic acid anhydrides; hydroxydicarboxylic acids; alpha-amino acids; beta-amino acids; gamma-amino acids; omega-amino acids; alpha-hydroxy carboxylic acids; beta-hydroxy carboxylic acids; gamma-hydroxy carboxylic acids, omega-hydroxy carboxylic acids; alpha-hydroxy aldehydes; beta-hydroxy aldehydes; gamma-hydroxy aldehydes; omega-hydroxy aldehydes; alpha-aldehyde carboxylic acids; beta-aldehyde carboxylic acids; gamma-aldehyde carboxylic acids; omega-aldehyde carboxylic acids; diamines; and hydroxy amines.

The second monomer preferably has multiple functional groups, that is the second monomer is multifunctional. Two functional groups that occur together on a monomer are herein termed as “a set of functional groups.” The second monomer has at least one set of functional groups selected from the list consisting of hydroxyl/carboxylic acid; carboxylic acid/carboxylic acid; hydroxyl/aldehyde; aldehyde/carboxylic acid; aldehyde/aldehyde; hydroxyl/hydroxyl; carboxylic acid/vinyl; amine/carboxylic acid; amine/amine; hydroxyl/vinyl/carboxylic acid; hydroxyl/olefin/carboxylic acid; olefin/carboxylic acid; carboxylic anhydride; carboxylic acid anhydride/hydroxyl; carboxylic acid anhydride/aldehyde; carboxylic acid anhydride/olefin; carboxylic acid anhydride/vinyl; carboxylic acid anhydride/amine; hydroxyl/olefin; hydroxyl/amine; aldehyde/olefin; aldehyde/vinyl; aldehyde/amine; aziridine; aziridine derivatives; epoxides; blocked isocyanates; colloidal silica; colloidal alumina; and combinations thereof.

An example of a second monomer with two or more functional group sets (wherein the set is the same) is: C—OH—(C)n-C—C—OH—(C)n-COOH (i.e., hydroxy/carboxylic acid).

Examples of the second monomer include acrylic acid; alcohols; aldehydes; glutaraldehyde; aspartamic acid; aspartame; lactic acid; salicylic acid; p-hydroxybenzoic acid; maleic acid; citric acid; sorbic acid; glycerin acid; alanine; glutamic acid; primary amines; carboxylic acids; dicarboxylic acid anhydrides; hydroxydicarboxylic acids; alpha-amino acids; beta-amino acids; gamma-amino acids; omega-amino acids; alpha-hydroxy carboxylic acids; beta-hydroxy carboxylic acids; gamma-hydroxy carboxylic acids; omega-hydroxy carboxylic acids; alpha-hydroxy aldehydes; beta-hydroxy aldehydes; gamma-hydroxy aldehydes; omega-hydroxy aldehydes; alpha-aldehyde carboxylic acids; beta-aldehyde carboxylic acids; gamma-aldehyde carboxylic acids; omega-aldehyde carboxylic acids; diamines; hydroxy amines; alpha-olefinic carboxylic acids; beta-olefinic carboxylic acids; gamma-olefinic carboxylic acids; omega olefinic carboxylic acids; alkylated acrylic acids; hydroxyalkylated acrylic acids; amino acrylic acid; aminoalkylated acrylic acids; alpha dimethylacrylic acid; beta dimethylacrylic acid; hydroxyacrylic acid, semialdehyde; ginipin; hydroxyethylmethylacrylate (HEMA); hydroxypropyl methylacrylate (HPMA); colloidal silica; colloidal alumina; epoxides; melamine, aziridines; carbodiimide; blocked di-isocyanates; blocked multiisocyanates; blocked di-thioisocyanates; and blocked multithioisocyanates. Examples of dicarboxylic acid anhydride are maleic anhydride or phthalic anhydride.

Vinyl groups, vis-à-vis olefin groups, occur as a terminal double bond. For example, hydroxy/vinyl/carboxylic acid is: C═C—C—OH—COOH; whereas, hydroxy/olefinic/carboxylic acid is: C—OH—C═C—COOH

For the purposes of the present specification, “blocked” isocyanates are reaction products of isocyanates with active hydrogen containing compounds which result in addition products having limited thermal stability. Blocking agents are disclosed for example in “Catalysis of Blocked Isocyanates with Non-Tin Catalysts”; Blank et. al.; King Industries, incorporated herein by reference. Suitable blocking agents include, for example, the malonates, triazoles, caprolactam, sulfite, phenols, ketoxims, pyrazols and alcohols. “Blocked” isocyanates that are suitable for the present invention have at least two reactive sites.

Colloidal silica suitable for the present invention are molecular units of silicon tetrahyroxyl Si(OH)₄ which form colloids or sols depending on various pHs. A typical size of the units is in the range of 1 to 5 nm.

In preferred embodiments, the multifunctional monomer component does not comprise a vinylsulfone or N-oxysuccinimide, or a sulfhydryl.

Solvent Compositions

Suitable solvent compositions are ones which can dissolve the multifunctional monomer component and saccharide component but which allow the formation of the network and do not alter or adversely impact the therapeutic properties of the composition. Preferably, the solvent composition comprises water, alcohols, alkylketones, arylalkylketones, ketoalcohols, cyclic ketones, heterocyclic ketones, ethers, cyclic ethers, esters and combinations thereof. Examples of suitable solvents include methanol, ethanol, propanol, isopropanol, butanol, methyl ethyl ketone, tetrahydrofuran, acetone, diacetone alcohol, N-methylpyrrolidone, dimethylsulfoxide, (DMSO) and dimethylformamide (DMF).

The multifunctional monomer component, saccharide component and the solvent composition form a reaction solution. Preferably, the solvent makes up from about 99.99 wt. % to about 50% wt. % of the reaction solution, more preferably, from about 99.89 wt. % to about 75% wt. % of the reaction solution.

Preferably, the saccharide component makes up about 0.01 wt. % to about 20 wt. % of the reaction solution, more preferably, about 0.1 wt. % to about 10 wt. % of the reaction solution.

Preferably, the multifunctional monomer component makes up about 0.001 wt. % to about 30 wt. % of the reaction solution, more preferably, about 0.01 wt. % to about 15 wt. % of the reaction solution.

In the reaction solution, preferably, the range of the ratio of the amount by weight of the saccharide component to the amount by weight of the multifunctional monomer component has a lower boundary of approximately 1:50. Examples of other lower boundaries include about 1:40; 1:30, 1:20; and 1:10.

Preferably, the range of the ratio of the amount by weight of the saccharide component to the amount by weight of the multifunctional monomer component has an upper boundary of approximately 10:1. Examples of other upper boundaries include about 5:1; 3:1; and 2:1.

For the non-leaching network compositions, preferably, the range of the ratio of the amount by weight of the first monomer to the amount by weight of the second monomer has a lower boundary of approximately 5:1. Examples of other lower boundaries include about 10:1; 20:1, and 25:1.

For the non-leaching network compositions, preferably, the range of the ratio of the amount by weight of the first monomer to the amount by weight of the second monomer has an upper boundary of approximately 100:1. Examples of other upper boundaries include about 50:1; 35:1; and 30:1.

For the controlled-leaching network compositions, the second monomer either is absent or is present at a low level. Examples of the amount of the second monomer in the leaching network compositions is about 3%, about 1%, about 0.5%, about 0.2%, about 0.1%, about 0.01%, about 0.001%, and about 0.0001% of the amount present in the non-leaching network compositions described above. The amount of the second monomer present in the network composition determines how readily the network disintegrates.

Linkages Between “Saccharide Component” and the Monomers

Upon removal of the solvent composition from the reaction solution, the multifunctional monomers and the functional group-containing saccharide component undergo unique linking activities.

The solvent can be removed by drying and/or evaporation. In some embodiments, the composition can also be cured. Curing is an accelerated evaporation of the solvent composition at elevated temperatures allowing the network to form faster and more completely, i.e., without leaving residual polymerizable starting molecules in the network. Preferably, the compositions are dried and cured at temperatures of about 20° C. to 180° C. for about 20 minutes to about 2 hrs, more preferably at about 60° C. to about 140° C.

Linkages form between the first monomer and the saccharide component, between the second monomer and the saccharide component, between the first monomers, between the first monomer and the second monomer, between the second monomers, and between the saccharide components. Examples of the linkages formed in the network composition follow.

The first monomer can form up to two linkages. The first monomer is linked to the saccharide component by ester linkages; ether linkages; amide linkages; ketone linkages; or combinations thereof.

The second monomer can form more than two linkages. The second monomer is linked to the saccharide component by ester linkages; ether linkages; amide linkages; ketone linkages; urea linkages; carbamate linkages, linkages originated from vinyl or olefinic groups, aluminum oxide linkages; siloxane linkages; or combinations thereof.

The first monomers are linked to each other by ester linkages; ether linkages; amide linkages; ketone linkages; or combinations thereof.

The first monomer is linked to the second monomer by ester linkages; ether linkages; amide linkages; ketone linkages; urea linkages; carbamate linkages; aluminum oxide linkages; siloxane linkages or combinations thereof.

The second monomers are linked to each other by ester linkages; ether linkages; amide linkages; ketone linkages; polyvinyl linkages, polyolefin linkages, urea linkages; carbamate linkages, aluminum oxide linkages, siloxane linkages or combinations thereof.

The saccharide components are linked to each other by ester linkages; ether linkages; amide linkages; ketone linkages; or combinations thereof.

The network compositions of the present invention form without an initiator, e.g., without the use of a photoinitiator or a chemical initiator. Examples of initiators are independent compounds, pendent chemical groups and photoinitiators used to promote coupling of monomers or polymers, such as free radical polymerization.

As a whole, the network compositions are composed of saccharide based chains. The chains comprise at least one saccharide component (SC), as defined above. A saccharide component is linked to at least one first monomer (M1), or at least one second monomer (M2), or various combinations of a first monomer and second monomer, as defined above.

In addition to linking to a saccharide component, the first and second monomers can link to other monomers. For example, the monomers can form a string of monomers from about 2 to about 10 monomers, more typically from about 3 to about 6 monomers.

The saccharide components and the monomers have an arbitrary order in the chains. For example, a chain in the network can be: SC-M1; or SC-M1-M1, or M1-SC-M1, or SC-M1-M1-M1, or M1-SC-M1-M1-M1; or M1-SC-M1-M1-M1-M2 or M2-M1-SC-M1-M1-M1 or M1-SC-M1-M1-M1; or SC-M1-M1- . . . M1-SC; or M1-SC-M1- . . . M1-SC-M1-Mi; or M1- . . . M1-SC-M2- . . . M1-M1-SC-M1-M2-SC-M1- . . . M1-M1; or M1-SC-M1-SC-M1-CS-M1; or M1-M1-SC-M1-SC-M1-M1-SC-M1 . . . ; or . . . M1-M1-M2-SC-M1-SC-M1-M1-M2-M1-SC-M1-M1-SC-M2-M2 . . . The length of the chains is not critical. A chain can comprise from about two (e.g., SC-M1) to about hundreds of M1, M2 and SC units.

The chains of the composition are cross-linked to one another by first and/or second monomers. Thus, the compositions form a complex network. The first monomer can form preferably up to two linkages; whereas, the second monomer can form preferably more than two linkages. In one embodiment, the most dominant link in the network is: SC-M1-SC. In another embodiment, the most dominant link in the network is: SC-M1-M1.

An example of a portion of a network follows

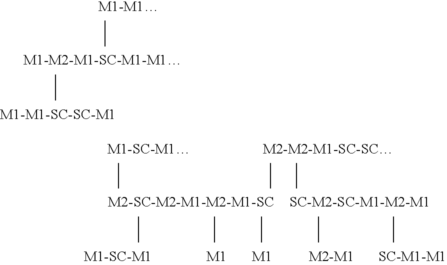

Applications of Composition

The network compositions of the present invention have qualities that allow its use in various fields. Due to surprising biopolymeric (i.e., biologically active) properties, the compositions are particularly useful in the medical field.

For example, the compositions exhibit the following novel cell-biological properties: non-cytotoxicity, anti-thrombogenicity, non-hemolytic properties, antimicrobial properties, anti-inflammatory properties, and anti-mitotic properties. The compositions retard cell proliferation in a desired controlled way of a healing process. For instance, the compositions exhibit significant retardation of smooth aortic muscle cell mitosis without the use of active drug components. The compositions exhibit their cell-biological properties without the addition of an eluding or bound drug or pharmaceutical. That is, these properties are intrinsic to the compositions.

The examples demonstrate some of the biologically active properties of the network compositions. For instance, Examples 9 and 43 demonstrate that the network compositions elicit no hemolysis with actual blood over 7 hrs. Examples 22 and 23 show that the compositions have cell proliferation retardation properties. Example 45 demonstrates evidence of platelet adhesion prevention. Accordingly, devices coated with compositions of the present invention can be used for vulnerable plaque prevention.

The compositions are particularly useful for coating substrates (i.e., objects) used in the medical field, i.e., medical devices. The compositions adhere to substrates made of polymeric and metallic materials, such as, for example, stainless steel, Nitinol®, plastics, polymers, glass, ceramics, cellulose fibers, synthetic fibers, textiles and alike.

In general, the compositions have a high degree of adherence to substrates. For example, polyurethane devices coated with the network compositions do not peel away from the devices even after five months of storage in water. Optionally, a primer coating can be used in some embodiments for better adhesion on critical substrates such as teflon, electropolished stainless steel, PEBAX and alike.

Substrate surfaces do not require pretreatment before the coatings of the present invention are applied. For example, the surfaces do not require acrylic acid in a surface grafting step under electron beam activation conditions, and they do not require corona or plasma treatment.

The compositions are lubricious, i.e., slippery. Once applied, the compositions form a lubricious film or coating on a substrate. The film reduces the coefficient of friction of the substrate by at least about 75%, at least about 80%, at least about 85%, at least about 95%, or at least about 97%.

Additionally, the coatings surprisingly do not have a tacky intermediate state during drying once exposed to bodily fluids, as usually observed with coatings of prior art. This is particularly noteworthy for peripherally inserted and applied medical devices. Devices inserted and left in place for extended periods of time without developing tack, or insignificant tack, substantially reduces the pain and discomfort of the patient.

A medical device is any object having surfaces that contact tissue, blood or other bodily fluids in the course of its operation, which fluids are subsequently used in a living body. Medical devices in which the network composition of the present invention can be incorporated include, but are not limited to, surgical implants, prostheses, and any artificial part or device which replaces or augments a part of a living body or comes into contact with bodily fluids, particularly blood. The objects can be in any shape or form including tubular, sheet, rod and articles of proper shape. Various medical devices and equipment usable in accordance with the invention are known in the art. Examples of devices include catheters, suture material, tubing, and fiber membranes. Examples of catheters include dialysis catheters, central venous catheters, thoracic drain catheters, angioplasty balloon catheters. Examples of tubing include tubing used in extracorporeal circuitry, such as whole blood oxygenators. Examples of membranes include polycarbonate membranes, haemodialysis membranes, membranes used in diagnostic or biosensor devices. Also included are devices used in diagnosis, as well as polyester yarn suture material such as polyethylene ribbon, and polypropylene hollow fiber membranes.

Further illustrations of medical devices include the following:autotransfusion devices, blood filters, blood gas exchange devices, blood pumps, blood temperature monitors, bone growth stimulators, breathing circuit connectors, bulldog clamps, cannulae, grafts, implantible pumps, impotence and incontinence implants, intra-occular lenses, leads, lead adapters, lead connectors, nasal buttons, orbital implants, cardiac insulation pads, cardiac jackets, clips, covers, dialators, dialyzers, disposable temperature probes, domes, drainage products, drapes, ear wicks, electrodes, embolic devices, esophageal stethoscopes, fracture fixation devices, gloves, guide wires, hemofiltration devices, hubs, intra-arterial blood gas sensors, intracardiac suction devices, intrauterine pressure devices, nasal spetal splints, nasal tampons, needles, ophthalmic devices, oxygenators (both sheet and tubular forms of membrane oxygenators), PAP brushes, periodontal fiber adhesives, pessary, pins, retention cuffs, screws, sheeting, sponges, staples, stomach ports, surgical instruments, transducer protectors, ureteral stents, vaginal contraceptives, valves, vessel loops, water and saline bubbles, achtabular cups, annuloplasty ring, aortic/coronary locators, artificial pancreas, balloons, batteries, bone cement, breast implants, cardiac materials, such as fabrics, felts, films, markers, mesh, patches, cement spacers, cochlear implant, defibrillators, generators, orthopedic implants, pacemakers, patellar buttons, penile implant, pledgets, plugs, plates, ports, prosthetic heart valves, sheeting, shunts, stylets, umbilical tape, valved conduits, and vascular access devices.

Due to their anti-coagulation properties and cell proliferation retardation properties (retardation of cell mitosis), the compositions of the present invention are particularly applicable to stents. A stent, for the purposes of this specification, is any device capable of being delivered by catheter. For example, a stent is any device capable of initially keeping a blood vessel open by physical means once inserted and expanded at an occluded side of the vessel. Stents include balloon-expandable and self-expanding stents. The balloon-expandable stent includes the metallic coils and slotted tube designs.

Drug eluding stents of the prior art have shown drawbacks by causing late thrombosis due to toxic side effects of the released drug. The network compositions of the present invention show a dual function of blood coagulation prevention and retardation of cell mitosis. Both properties are vital for keeping the blood vessel open by preventing blood clot formation and retarding cell proliferation.

Examples of medical devices particularly useful with the present invention include non-expendable or expendable stents, catheters, guide wires, shunts, screws, pins, prostheses, plates, films, sponges, sutures, medical tubings, cannulas, balloons, needles, markers, stylets, surgical rods, guidewire tubes, coiled guiding tubes, coiled catheters, electrodal coils, blades, fibers, wound dressing fibers, band aids, suture threads, ocular lens delivery device, ocular lenses, ocular catheters, dialysis catheters, wound drains, and bone implants.

The network compositions are applied to the object by dipping, spraying, flooding, foaming, roll-coating, dipping, brushing, electrolytic depositing, electrostatic spraying, electroplating, vacuum treatment, pressure treatment, vac-vac, or combinations thereof. The compositions can be applied in layers with or with out a primer or an additional sealing topcoat. Preferred primer and topcoat formulations are described in U.S. Pat. No. 4,642,267 and U.S. Pat. No. 7,008,979, both of which are incorporated herein by reference in their entireties.

Additionally, the compositions can be layered during or after previous layers are dried and cured. The compositions are also suitable for the combination with commonly used primers for critical substrates, such as electro polished stainless steel. The network compositions of the present invention also show good compatibility with coating layers based on polyurethane/polyvinyl pyrrolidone technology.

In additional to coating the outside surfaces of medical devices, the readily processed curing of the present invention allows application of coatings to the inner lumens of medical devices.

Depending on the specific application, the thickness of the composition can be from about 0.1 microns or thicker.

In a preferred embodiment, the saccharide component of network composition is heparin. In another embodiment, a coating made of any of the network compositions of the present invention is grafted with a heparin composition. These heparin coatings have non-coagulating properties without leaching. Such coatings are particularly beneficial with stents.

In another embodiment, a conventional primer is first applied to a substrate, followed by a layer of the network composition containing heparin, and then sealing with a network composition containing chitosan.

In further preferred embodiment, the saccharide component of the network composition is chitosan. For example, a chitosan based coating according to Example 48 demonstrated prevention of E. coli and P. aruginosa colonization over a two week period.

The network compositions are also used to coat products which benefit from the benign antimicrobial surface activity of the compositions. Examples of such products include textiles, cellulosics, and fiber containing feminine hygiene products. For example, tampons coated with the network compositions improve the hygienic performance of the products. The network compositions can also be used to coat the textiles of bandages, swabs, gausses and other surgical tools, including gloves. For example, a bandage coated with these network compositions placed on a wound minimizes scar formation.

The compositions can also be used to coat orthopedic implants to afford anti-microbial protection and to prevent inflammation in a benign way.

In another embodiment, the compositions can also be used therapeutically on a stand alone basis, i.e., the compositions can be directly applied to, for example, wounds. For instance, applying the composition on a wound (e.g., a cut) suppresses bleeding and prevents scar formation.

In addition to the suitability for general medical devices, the network compositions also can be used in veterinary applications, in particular, with dairy animals. For example, the network compositions can be used for the prevention and treatment of infections of a dairy cow uterus after giving birth, or for the prevention and treatment of mastitis. In such applications, the composition is preferably in the form of a foam.

In one embodiment, the network compositions are non-leachable. Thus, the compositions retain their integrity for extended periods of time. For instance, Example 48 demonstrates that the non-leachable network compositions significantly prevent microbial colonization for up to 2 weeks. Leachable antimicrobial actives would lose activity after 3 to 4 days.

In another embodiment, the network compositions are subject to hydrolytic leaching. These networks disintegrate when exposed to phosphate buffered saline (PBS) aqueous solutions in a programmable time frame. The amount of the second monomer present in the network determines how readily the network disintegrates.

The preferred saccharide component used in the leaching networks is chitosan. Along with chitosan, salicylic acid (i.e., hydrolysis product of aspirin), or lactic acid and aspirin, are preferably the first monomers. Other preferred saccharide components are glucoseamine and hyaluronic acid. When such leaching networks are exposed to bodily fluids, the networks disintegrate and release the components of the network into the body. Such a mode of action is referred to as a “slime-off” mode of action.

Additional Optional Ingredients

Although the compositions are biologically active on their own, in some embodiments, the network compositions can further comprise additional optional ingredients, such as film-improving ingredients, biologically active materials or a combination of both. The additional optional ingredients can be added at any level necessary to achieve the purpose of the ingredient. A preferred level is about 0.001 wt % to about 3 wt % of the reaction solution.

Examples of film-improving ingredients include surfactants, wetting agents, plasticizers, humectants, viscosity modifiers, defoamers, emulsifiers, dyes, pigments, colorants, UV absorbers, radical scavengers, antioxidants, anti-corrosion agents, carbon dioxide releasers, optical brighteners, fluorescers, bleaches, bleach activators, bleach catalysts, non-activated enzymes, enzyme stabilizing systems, chelants, coating aids, metal catalysts, metal oxide catalysts, organometallic catalysts, film forming promoters, hardeners, linking accelerators, flow agents, leveling agents, lubricants, matte particles, rheological modifiers, thickeners, electrolytes, conductive or non-conductive metal oxide particles, colloidal antimicrobial metal oxides, magnetic particles, anti-static agents, pH control agents, perfumes, preservatives, biocides, esticides, anti-fouling agents, algicides, bactericides, germicides, disinfectants, fungicides, bio-effecting agents, vitamins or combinations thereof. A preferred example of an electrolyte is lithium chloride and magnesium acetate.

The compositions of the invention are useful as carriers for a wide variety of biologically-active substances having curative or therapeutic value for human or non-human mammals. In one embodiment, the biologically active material is grafted or linked to the composition by chemical functional group interaction for localized treatment. In another embodiment, the biologically active material is physically embedded to enable controlled release.

Examples of biologically active material include antithrombotic agents, biostatic agents, cytostatic agents, radiation emitters, pharmaceuticals, biomolecules, anti-inflammatory agents, immunosuppressants, antibiotics, antiseptics, hypnotics, sedatives, tranquilizers, anti-convulsants, muscle relaxants, analgesics, antipyretic agents, local anesthetics, antispasmodics, antiulcer agents, antivirals, antibacterials, anti fungals, sympathomimetic agents, cardiovascular agents, antitumor agents, and combinations thereof. Biologically-active materials are added in pharmaceutically-active amounts.

For example, the network compositions serve as carriers for fast coagulating peptides in order to stop bleeding within about 15 seconds. Such peptides are known in the art (Rutledge Ellis-Behnke).

The optional ingredients can be initially mixed with any of the three components before placing all three of the components together, that is, the optional ingredient can be initially mixed with the solvent composition or the multifunctional monomer component or the saccharide component. Preferably, water-soluble ingredients are initially mixed with the solvent composition. Preferably, water-insoluble additives are initially mixed with the saccharide component. Additives may also be applied to the endproduct network composition.

The present invention also includes methods of treatment with the leaching and non-leaching networks of the present invention. Conditions that can be treated with the present invention include any conditions that can be treated with the saccharide component, first monomer or second monomer. For example, any condition that can be treated aspirin or heparin can be treated with the compositions of the present invention, including, for example, pain and embolisms.

### EXAMPLES

The following non-limiting examples have been carried out to illustrate preferred embodiments of the invention. These examples include the preparation of coating compositions according to the invention, analysis of the coatings and testing of the coatings.

### 1. TEST METHODS

Physical Testing

Viscosity Test

All measurements were performed with a Brookfield RVDV II+rotational viscometer available from Brookfield Engineering Labs, Inc., Stoughton, Mass., USA. The recommended procedure is followed, with the following exceptions. The recommended procedure is varied by using a smaller vessel and removing the guard leg. The calibration is to be determined using a 600 ml low form griffin type beaker with Glycerin (1400 cp) and olive oil (80 cp) at 100 RPM. All subsequent measurements are performed in 50 ml beakers at 100 RPM with the appropriate spindle.

Contact Angle

As used herein, the term “hydrophilic” describes surfaces which are wetted by DI water deposited onto the surface. The state of the art respecting wetting of materials allows definition of hydrophobicity (and wetting) in terms of contact angles and the surface tension of the liquids and solids involved. This is discussed in detail in the American Chemical Society Publication entitled “Contact Angle, Wettability, and Adhesion edited by Robert F. Gould and copyrighted in 1964.

The test for determining the contact angle was conducted by wetting polycarbonate as a representative surface. Water as the representative liquid was placed on the representative surface. The contact angle between the liquid and the surface is less than 90° or when the liquid will tend to spread spontaneously across the surface. Both conditions normally coexisting. The water is brought on to the surface to be tested by a syringe needle. Method and read-out was conducted according to the CAM-MICRO equipment supplied by Tantec, Inc. This test was used as general evaluation criteria for formulations of mentioned examples and comparative examples to determine the hydrophilic properties of compositions of the present invention. This method is suitable for evaluating hydrophilic coating properties in medical applications.

Application of Compositions

Examples of compositions of the present invention and comparative examples were usually applied by dipping, brushing, wiping, spray-coating, foaming, foam coating, electrolytic depositing or by a roller for general coating or by a wire bar coating for specific coating thickness.

Uniformity/Hydrophilic Properties

To check the even distribution of a hydrophilic coating the staining test with an aqueous solution of FD&C Blue is conducted by dipping the coated sample into the solution. In some cases a toluidine blue (0.005 to 0.03% for heparin detection) or other food color solution were used for evaluating the uniformity of the coating.

The preferred uniformity test for medical coatings are conducted with crystal violet solution.

Durability Testing

Durability tests were conducted primarily in conjunction with the friction reduction tester of standard computerized recording. Byk Gradner supplies equipment and test description which was used for evaluating the abrasion resistance of hydrophilic coatings. Test method 18.1.1 of catalog 90 allows variations regarding rubbing force, rubbing tool (brush or sponge), number of rubbing cycles with or without water. Cycles usually run between 20 and 100 with evaluation stop every one to two cycles. After the abrasion test the remaining coating becomes visible by staining it with the crystal violet solution. The estimated % degree of non-stained area allows relative conclusions regarding the improvement of durability of the coating.

% Friction Reduction/Kinetic Friction Reduction

The tester consists of a friction machine and a computer. The pull with which a sled is dragged over a coated surface with or without water contact is recorded and compared in a chart with the uncoated sample. The tester allows automatic data collection with Zero setting. The sled further may contain a foam pad. The wetted test samples are pulled according to settings and pulling forces which are recorded by a computer print-out chart. Formulation improvements of lubricity of coatings or low residual friction of hydrophilic coatings for medical devices according to the present invention reveal. The coating was tested in reference to ASTM D 1894-87 Standard Test Methods for Static and Kinetic Coefficients of Friction of Plastic Film and Sheeting.

Adhesion Test

Coated substrate according to the present invention are scribed by 5×5 cross cuts. An adhesive tape 3M Type 610 is firmly pressed onto the cuts and peeled of. The degree of coating peel-off is used in a relative comparison of improved compositions of the present invention. Adhesion of medical coating can be evaluated.

Immersion Weight Gain Test

Coatings of various compositions were dried at room temperature over night or cured at 70° C. for 10 minutes and checked for their water uptake capacity by determining the weight differences between known compositions and compositions of the present invention before and after immersion in water. This test applies primarily to the drug loading capacity.

Biological Testing

Anti-Microbial Testing

The coatings of the present invention were tested for their antimicrobial activity by a laboratory test method, which provides a qualitative and semi-quantitative procedure for the evaluation of antimicrobial activity by diffusion of the antimicrobial agent through agar. The method is derived from the “Parallel Streak Method” which is based on the Antibacterial Activity Assessment of Textile Materials; AATCC Test Method 147-1998.

The cultures were prepared fresh overnight. The organisms were incubated with Tryptone Soy Broth (TSB) at 37.degree. C. the day before the test. The bacterial cell suspension in TSB was >10. sup.7 cells per ml. On the day of test, hot agar samples were cooled in sterile tubes and then 0.1 ml of the individual culture was added to the melted agar. The agar samples were poured onto plates after mixing, allowed to gel and then test samples of hydrogels of the present inventions were placed on top of the agar. Incubation was then continued for one and 5 days and the zone of inhibition of bacterial growth approximated around each sample.

Anti-Microbial Surface Testing

Polyurethane films were coated with the test formulations to test for the prevention of bacterial colonization. The films were retained in PBS at room temperature until leaching is complete. Substrates were than exposed to UV radiation for 5 minutes. Bacterial solutions were placed on the surfaces and covered with an agar plate. Samples then are examined for bacterial growth after incubation at 37 C for 24hrs or for fungal growth after 72 hrs using a 25× microscope equipped with a digital camera.

Cell Cytotoxicity Testing

Cytotoxicity testing was done as in vitro biocompatibility study based on the International Organization for Standardization (ISO10993) test “Biological Evaluation of Medical Devices”, Part 5. Coatings for tests were applied onto 18 mm glass cover slips and incubated with 2 ml L929 media for 24 hrs. Media was removed from the cells seeded for 24 hrs and replaced with the media from the disks. The cells were examined for cytotoxicity 72 hrs later and compared with the a negative control sample.

Assay To Assess Cell Proliferation

Coated glass cover slips (18 mm dia.) are placed into 12-well dishes. Cell are seeded into the wells at ˜20% confluence, media is added and the cells allowed to grow for 3-4 days @ 37° C. in a 5% CO₂ atmosphere. The glass cover slips are removed, rinsed in PBS and placed into a new 12-well plate along with 0.50 ml of fresh complete media. 25 ul of Cell proliferation reagent (MTS) is added and incubated at 37° C. for 1 to 3 hours. 50 ul of each is transferred to a 96-well plate and the absorbance at 492 nm recorded.

Test for Surface Leaching of Anti-coagulative Coating

In order to determine if all non-bound anti-coagulative coating was washed from the coated films, the films were soaked in 1 ml of PBS for 24 hrs at RT. 50 ul of this PBS was incubated with 100 ul citrated human plasma at 37 C for 2 min. The clotting mechanism was stimulated by the addition of 200 ul of thromboplastin-DL and the time of the clot formation recorded. When a normal clotting time of ˜12 seconds was obtained, the film was ready for surface testing.

Testing for the Ability of an Anti-Coagulative Surface to Inhibit Whole Blood Clotting

100 ul of citrated human whole blood was incubated with 100 ul of thromboplastin-DL on the anticoagulative surface. The time of clot formation was recorded and photographs taken. All liquid was removed from the wells and a second photograph was taken illustrating the formation of the clot.

Testing for the Ability to Inhibit Prothrombin Time

1 ml of citrated human whole blood was incubated in a microfuge tube containing a coated polyurethane film (0.50×2 cm) for 30 minutes at 37 C. The whole blood was centrifuged and the plasma tested for prothrombin time according to manufacture's instructions using a fibrin timer.

Toluidine Blue Staining

A solution of toluidine blue stain (0.05%) was applied in an amount to cover the PU film and the incubation continued for 1 min. The film was washed several times and a photograph taken to show the heparin staining.

Hemolysis Assay

A 100 mg piece of gel was added into 1 ml of citrated human whole blood. It was incubated for 30 min at 37 C. and then the whole blood spun at 1000×g for 5 minutes. The supernatant (plasma) was removed and the absorbance read at 600 nm vs a saline or water blank. The plasma was diluted with saline if necessary to get a reading between 0.5 and 1.0.

Assays for Measuring Platelet Adhesion

Glass cover slips (18 mm diameter) were coated.

Platelets were isolated from platelet-rich plasma by centrifugation or ADIAgel platelet separation tubes. Platelets were adhered to glass, fixed and stained for visualization using fluorescein-labeled DIOC6.

### 2. EXAMPLES

### Example 1

5 g of Chitosan were mixed with 15 g of Lactic Acid into 100 g of Isopropyl alcohol, 25 g Tetrahydrofuran, 25 g N-methylpyrrolidone, 0.1 g Wetting agent FC-170, 329.40 g water and 0.5 g acrylic acid. The coating was applied to a silicone tubing. After curing at 120 C for 30 min the coating showed good lubricity and durability according to the method described above. No primer was required for the test results obtained. The friction of a wet non-coated unit was recorded with 0.45 lbs of pulling force. In comparison a wet coated unit with 0.045 lbs pulling force revealed a friction reduction of 90% for the first cycle. Additional cycles of up to 50 showed insignificant changes of the friction reduction. A slight additional friction reduction even was noticed at cycle 50 with an overall average of about 91%.

### Example 2

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. The coating was applied to a polyurethane catheters. After curing at 120 C for 30 min the coating showed good lubricity and durability according to the method described above. The friction of a wet non-coated unit was recorded with 0.87 lbs of pulling force. In comparison a wet coated unit with 0.07 lbs pulling force revealed a friction reduction of 92% for the first cycle. Additional cycles of up to 50 showed further friction reduction with a friction reduction for cycle 10 to cycle 50 of about 97%.

### Example 3

4 g Chitosan are dissolved in 20 g Isopropanol, 10 g NMP, 10 g THF and 140 g of deionized water with 16 g of Lactic Acid. A polyurethane tubing was dipped into the solution. The formulation forms a film upon drying and curing for 15 min at 120 C. When contacted with water the film becomes lubricious. According to the method above the coated tubing was evaluated for friction reduction. A friction reduction of over 85% was detected for the first cycle, however, durability was compromised after short time letting the friction reduction drop below 85%.

### Example 4

5 g of Chitosan were mixed with 25 g of Lactic Acid into 100 g of Isopropyl alcohol, 25 g Tetrahydrofuran, 25 g N-methylpyrrolidone, 0.1 g FC-170, 319.40 g water and 0.5 g acrylic acid. The coating was applied to a polyurethane catheters. After curing at 120 C for 30 min the coating showed good lubricity and durability according to the method described above. The friction of a wet non-coated unit was recorded with 0.87 lbs of pulling force. In comparison a wet coated unit with 0.06 lbs pulling force revealed a friction reduction of 93% for the first cycle. Additional cycles of up to 50 showed further friction reduction with a friction reduction for cycle 10 to cycle 50 of about 97%.

### Example 5

### Comparative Example 2 from U.S. Pat. No. 4,662,267)

To 47 g of water and 10 g N-methylpyrrolidone was added 10 g of polyvinylpyrrolidone and 33 g of linear polyurethane aqueous dispersion. Films cast from the resulting viscous dispersion were lubricious when wet (coefficient of friction 0.08) and imbibe water forming elastic, transparent films useful as burn and wound dressings. The solution can also be used to spin fibers which are tough and elastic when wet and can be used to produce hydrophilic foams via either mechanical frothing or casting films with added acetone and drying with heat in vacuum.

### Example 6

1 g Chitosan and 3 g Lactic acid was mixed into 20 g IPA, 65.88 g Water, 5 g Tetrahydrofuran 5 g N-Methylpyrrolidone, 0.02 g FC-170 and 0.1 g Acrylic Acid. The formulation was used to dipcoat a silicone catheter and then cured for 30 min at 120 C.

The formulation forms a stable network upon evaporation of the carrier solvents and becomes lubricious upon contact with saline solution.

The coating was left in saline solution for 24 hrs without getting removed from the device surface showing good stability and adhesion.

The coated device was tested for its friction reduction according to the above mentioned method.

Uncoated devices have a required pulling force of routinely 0.3 lbs. The coating caused in the wet state to degrease the friction to a pulling force of 0.02 lbs being a friction reduction of over 93%.

### Example 6a

1% of Chitosan were mixed with 3% g of Lactic Acid into 20% of Isopropyl alcohol, 5% Tetrahydrofuran, 5% N-methylpyrrolidone, 0.02% FC-170, 65.88% water and 0.1% maleic anhydride. The coating was applied to polyurethane tubing. After curing at 100 C for 1 hr the coating showed good lubricity and durability to the touch in comparison to example 6 when contacted with water.

### Example 6b

Formulation of Example 6a was diluted 50% with water and used in the diluted form as before. The coating was applied to polyurethane tubing. After curing at 100 C for 1 hr the coating showed good lubricity and durability to the touch in comparison to example 6 when contacted with water.

### Example 6c

3% of heparin sodium salt were mixed with 15% of Lactic Acid into 82% water with 0.3% Maleic anhydride. The coating was applied to polyurethane tubing on top of the cured coating of Example 5. After curing at 100 C for 1 hr the coating showed good uniform staining with aqueous Toluidine Blue solution.

### Example 6d

1% of Chitosan were mixed with 3% g of Lactic Acid into 20% of Isopropyl alcohol, 5% Tetrahydrofuran, 5% N-methylpyrrolidone, 0.02% FC-170, 65.88% water and 0.1% 2-hydroxyethyl methylacrylic acid (HEMA). The coating was applied to polyurethane tubing. After curing at 100 C for 1 hr the coating showed very lubricious property by the finger test comparable to Example 6 when contacted with water.

### Example 6e

3% of heparin sodium salt were mixed with 15% of Lactic Acid into 82% water with 0.3% 2-hydroxyethyl methylacrylic acid (HEMA). The coating was applied to polyurethane tubing on top of the cured coating of Example 5. After curing at 100 C for 1 hr the coating showed good uniform staining with aqueous Toluidine Blue solution.

### Example 6f

### Wet-Tack Adhesion

2% Chitosan were mixed with 10% Lactic Acid into 30% of Isopropyl alcohol, 3% PEG-400, 55% water. The gelly coating was applied to a release liner and transfered onto a piece of wet liver fixed in a release force measuring/recording device. A second piece of wet liver was put on top and the pieces held together for 5, 30 and 60 min respectively. Pulling forces of up to 190 g within the first 1 to 3 sec were detected.

### Example 7

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. The coating was applied to polyurethane cladded guidewire. After curing at 120 C for 30 min the coating showed good lubricity and durability according to the method described above. The friction of a wet non-coated unit was recorded with 0.268 lbs of pulling force. In comparison a wet coated unit with 0.021 lbs pulling force revealed a friction reduction of 92% for the first three cycles Additional cycles of up to 25 showed further friction reduction with a friction reduction better than 97%.

### Example 7a

0.5 g of Chitosan were mixed with 1.5 g of Lactic Acid into 10 g of Isopropyl alcohol, 2.5 g Tetrahydrofuran, 2.5 g N-methylpyrrolidone, 0.01 g FC-170, 83 g water and 0.05 g acrylic acid. The coating was applied to PVC tubing. After curing at 100 C for 30 min the coating showed similar lubricity and durability to the touch in comparison to example 7 when contacted with water.

### Example 7b

0.5 g of Chitosan were mixed with 2.5 g of Lactic Acid into 20 g of Isopropyl alcohol, 5 g Tetrahydrofuran, 5 g N-methylpyrrolidone, 0.02 g FC-170, 66 g water and 0.1 g acrylic acid. The coating was applied to PVC tubing. After curing at 100 C for 30 min the coating showed similar lubricity and durability to the touch in comparison to example 7 when contacted with water.

### Example 7c

1 g Chitosan and 3 g Lactic acid was mixed into 20 g IPA, 66 g Water, 5 g Tetrahydrofuran, 5 g N-Methylpyrrolidone, 0.02 g FC-170, 2.5% colloidal silica Nycol DP-5110 and 0.1 g Acrylic Acid. The formulation was used over a standard primer to topcoat twice a polyurethane cladded guidewire and then cured for 30 min at 90 C and 10 min at 120 C.

The formulation forms a stable network upon evaporation of the carrier solvents and becomes lubricious upon contact with saline solution.

The coated device was tested for its friction reduction according to the above mentioned method. An uncoated devices has a required pulling force of 0.24 lbs. The coating caused in the wet state to degrease the friction to a pulling force of 0.015 lbs being a friction reduction of almost 94%.

### Example 8

### Comparative Testing

Comparative Example from U.S. Pat. No. 4,662,267)

A typical solvent based formulation according the U.S. Pat. No. 4,662,267 showed only a friction reduction of 86% for the first 3 cycles and a friction reduction of 88% for the following 25 cycles.

### Example 9

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. Glass disks, 18 mm in diameter were dip-coated and cured for 30 min at 120 C.

2 ml of citrated human whole blood was incubated for up to 7 hours at 37 C in the presence of the coated glass cover slips. 0.5 ml was centrifuges and the absorbance of a 1:20 dilution in PBS was recorded at specific time intervals.

Conclusion: Over 3 and 7 hours the human blood cells did not experience hemolysis evidenced by the practically stable visible light absorption at 600 nm ranging from about 0.125 to 0.15 relative intensity. The formulation has no hemolytic effect.

### Example 10

5 g of HCMF Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. The coating was applied to a polyurethane catheters. After curing at 120 C for 30 min the coating showed good lubricity and durability according to the method described above. The friction reduction was still 90% after 25 cycles.

### Example 11

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. The coating was applied to a polyurethane cladded guide wire. After curing at 120 C for 30 min the coating showed good lubricity and durability according to the method described above. The friction reduction was still 94% after 25cycles.

### Example 12

### Comparative Sample of U.S. Pat. No. 7,008,979

To 281 g of water was added a solvent mix of 89 g which consisted of isopropanol and diacetone alcohol, 19 g polyvinylpyrrolidone solution (20% of Kollidone K90, BASF), 19 g of aqueous aromatic polyurethane dispersion NeoRez R-940 (NeoResins), 0.8 g aziridine crosslinker NeoCryl CX 100 (Zeneca Resin) and 11 g aqueous colloidal silica solution N5110 (Eka-Akzo). The hydrophilic formulation was mixed and revealed good shelf life. Wet friction on a PU cladded guide wire showed comparative friction rediction of 95% after the first 3 cicles and 94% after 25 cycles.

### Example 13

1 g of Chitosan were mixed with 2 g of Aspirin into 20 g of Isopropyl alcohol, 5 g Tetrahydrofuran, 5 g N-methylpyrrolidone, 0.02 g FC-170, 68.98 g water. The coating was applied to a polyurethane tubing. After curing at 120 C for 30 min the coated tubing was placed in water and remained lubricious for 5 v months without getting removed.

### Example 14

5 g Chitosan are dissolved in 100 g Isopropanol, 25 g NMP, 25 g THF and 329.4 g of deionized water with 15 g of Lactic Acid and 0.5 g Acrylic Acid. Two polyurethane tubings were dipped into the solution. The formulation forms a film upon drying and curing for 20 min at 80 C and was compared at pH 6 with the second dip-coated tubing cured at 120 C for 20 min. When contacted with water the film becomes lubricious. According to the method above the coated tubing was evaluated for friction reduction. The friction reduction was improved by 89% with the 120 C curing versus the sample cured at 80 C.

### Example 15

5 g Chitosan are dissolved in 100 g Isopropanol, 25 g NMP, 25 g THF and 329.4 g of deionized water with 15 g of Lactic Acid and 0.5% Acrylic Acid. Two polyurethane tubings were dipped into the solution. The formulation forms a film upon drying and curing for 20 min at 80 C and was compared at pH 8 with the second dip-coated tubing cured at 120 C for 20 min. When contacted with water the film becomes lubricious. According to the method above the coated tubing was evaluated for friction reduction. The friction reduction was improved by 75% with the 120 C curing versus the sample cured at 80 C

### Example 16

5 g Chitosan are dissolved in 100 g Isopropanol, 25 g NMP, 25 g THF and 329.4 g of deionized water with 15 g of Lactic Acid and 0.5% Acrylic Acid. A primer was applied first to one of two PU cladded guidewires. The primed vs non-primed coated wire cured at same condition, (20 min, 120 C) was compared. The primed unit showed a friction reduction of 93%. The non-primed unit with a friction reduction of 92.8% showed practically no difference in achieved friction reduction.

### Example 17

5 g Chitosan are dissolved in 100 g Isopropanol, 25 g NMP, 25 g THF and 329.4 g of deionized water with 15 g of Lactic Acid and 0.5% Acrylic Acid. Tubing of Polyurethane, Pebax and Silicone was coated in a comparative test. The formulation showed a friction reduction on polyurethane of 93% for the first cycle and almost 97% after 50 cycles. On Pebax a friction reduction of 95% for the first cycle and still 95% after 50 cycles.

### Example 18

5 g Chitosan are dissolved in 100 g Isopropanol, 25 g NMP, 25 g THF and 329.4 g of deionized water with 15 g of Salicylic acid and 0.5% Acrylic Acid. Tubing of polyurethane was coated with a primer cured at 100 C for 15 min and then top-coated with the modified formulation and cured again at 100 C for 1 hr. a friction reduction of 94% was achieved after 25 cycles.

### Example 19

1 g Chitosan is dissolved in 65.88 g DI water, 3 g of lactic acid, 20 g Isopropanol, 5 g NMP, 5 g THF and 0.02 g FC170 and 0.3% Acrylic Acid. PU cladded guidewire was coated, cured at 120 C for 20 min and tested at pH 6. a friction reduction of 85% was detected.

### Example 20

1 g Chitosan is dissolved in 65.91 g DI water, 3 g of lactic acid, 20 g Isopropanol, 5 g NMP, 5 g THF and 0.02 g FC170. PU cladded guidewire was coated, cured at 120 C for 20 min and tested at pH 6. A friction reduction of 92% was detected.

### Example 21

1 g Chitosan is dissolved in 65.91 g DI water, 3 g of lactic acid, 20 g Isopropanol, 5 g NMP, 5 g THF and 0.02 g FC170, 0.1 g acrylic acid and 0.2% of 0.1 g of 2,2′-azobis(2-methyl-N-(2-hydroxyethyl)propionamide). PU cladded guidewire was coated, cured at 120 C for 20 min and tested at pH 6. A friction reduction of 92% was detected.

### Example 22

Influence on Retardation of Cell Proliferation—Mouse cells

Samples of 18 mm glass disks coated with formula according to example 3 were subject to cell proliferation test as outlined above. Cells were seeded in a 12 well tissue culture plate containing control (blank) and coated 18 mm glass cover slips. After 3 days of growth, the cover slips were rinsed in PBS and placed into a new 12-well dish containing 0.50 ml fresh media. 25 ul of MTS tetrazolium reagent was added and incubated for 3 hours at 37 C. Reactions were read in a 96-well plate at 492 nm.

Formulation according to example 3 had a 67% decrease of Murine L929 cell proliferation after 3 days in comparison to the control.

### Example 23

Influence on Retardation of Cell Proliferation—Smooth Muscle Cells

Samples of 18 mm glass disks coated with formula according to example 3 were subject to cell proliferation test as outlined above and tested according to example 3b.

After 3 days or 5 days of growth, the cover slips were rinsed in PBS and placed into a new 12-well dish containing 0.50 ml fresh media. 25 ul of MTS tetrazolium reagent was added and incubated for 3 hours at 37 C. Reactions were read in a 96-well plate at 492 nm.

Formulation according to example 3 had a 76% decrease of Human Aortic Smooth Muscle Cell proliferation after 3 days in comparison to the control. Surprisingly it was also found that the Human Aortic Smooth Muscle Cell Proliferation decreased even further after 5 days to 95% in comparison to the uncoated control

### Example 24

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. The coating was applied to a polycarbonate sheet and cured at 120 C for 30 min tested according to the above mentioned method for the cytotoxicity potential. After 72 hrs the samples were graded with zero having no reactivity and no cell lysis.

### Example 24

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. The coating was applied to a polycarbonate sheet and cured at 120 C for 30 min tested according to the above mentioned method for the cytotoxicity potential. After 72 hrs the samples were graded with zero having no reactivity and no cell lysis.

### Example 25

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. The coating was applied to a polyurethane films and put into growth medium together with suspended L929 cells. After 2 and 7 days the morphology of the cells attached to the surface was observed.

### Example 26

Anticoagulation Coating with Non-Leaching Heparin

A polyurethane film was primed and coated with the formula 5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by using a stainless steel wire drawdown rod#10. This layered system was cured for 30 min at 120 C. A 1% heparin formulation in water and 3% lactic acid was used as topcoat and applied with a stainless steel wire drawdown rod #10 and cured at 120 C for 30 min. The coating was washed/leached to remove any non bonded residual heparin by placing the film in 250 ml of water and place in a mechanical shaker overnight. According to the above described whole blood test the surface showed no blood coagulation.

### Example 27

Anticoagulation Coating with Non-Leaching Heparin—Optimization

A polyurethane film was primed and coated with the formula 5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by using a stainless steel wire drawdown rod#10. This layered system was cured for 30 min at 120 C. A 1%, 0.5%, 0.25%, 0.125% and 0% heparin formulation in water and 3% lactic acid was used as topcoat and applied with a stainless steel wire drawdown rod #10 and cured at 120 C for 30 min. The coating was washed/leached to remove any non bonded residual heparin by placing the film in 250 ml of water and place in a mechanical shaker overnight. According to the above-described whole blood test the surface showed the following non-coagulation potential: 1% Heparin: No clot, a 0.1% Toluidine Blue solution (TB) in PBS showed dark purple staining; 0.5% Heparin: No clot, TB showed dark purple staining; 0.25% Heparin: No clot, TB showed lighter purple staining; 0.125% Heparin: tiny clot, TB showed lighter purple staining; 0% Heparin: Clot, no TB staining

### Example 28

Anticoagulation Coating with Non-Leaching Heparin

A polyurethane film was primed and coated with the formula 5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by using a stainless steel wire drawdown rod#10. This layered system was cured for 30 min at 120 C. A 3% heparin formulation in water with 22% lactic acid was used as topcoat and applied with a stainless steel wire drawdown rod #10 and cured at 120 C for 30 min. The coating was washed/leached to remove any non bonded residual heparin by placing the film in 250 ml of water and place in a mechanical shaker overnight. According to the above-described whole blood test the surface showed no clot and a dark purple staining with TB.

### Example 29

Anticoagulation Coating with Non-Leaching Heparin—Control

A polyurethane film was primed and coated with the formula 5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by using a stainless steel wire drawdown rod#10. This layered system was cured for 30 min at 120 C. No heparin formulation was used for topcoat and applied with a stainless steel wire drawdown rod #10 and cured at 120 C for 30 min. The coating was washed/leached by placing the film in 250 ml of water and place in a mechanical shaker overnight. According to the above described whole blood test the surface showed blood coagulation.

### Example 30

Anticoagulation Coating with Non-Leaching Heparin—Control

A polyurethane film was primed and coated with the formula 5 g of Chitosan were mixed with 15 g of Salicylic Acid into 100 g of Isopropyl alcohol, 25 g Tetrahydrofuran, 25 g N-methylpyrrolidone, 0.1 g FC-170, 329.40 g water and 0.5 g acrylic acid by using a stainless steel wire drawdown rod#10. This layered system was cured for 30 min at 120 C. No heparin formulation was used for topcoat and applied with a stainless steel wire drawdown rod #10 and cured at 120 C for 30 min. The coating was washed/leached by placing the film in 250 ml of water and place in a mechanical shaker overnight. According to the above described whole blood test the surface showed blood coagulation.

### Example 31

Anticoagulation Coating with Non-Leaching Heparin

A polyurethane film was primed and coated with the formula 5 g of Chitosan were mixed with 15 g of Salicylic Acid into 100 g of Isopropyl alcohol, 25 g Tetrahydrofuran, 25 g N-methylpyrrolidone, 0.1 g FC-170, 329.40 g water and 0.5 g acrylic acid by using a stainless steel wire drawdown rod#10. This layered system was cured for 30 min at 120 C. A 1% heparin formulation in water and 3% lactic acid was used as topcoat and applied with a stainless steel wire drawdown rod #10 and cured at 120 C for 30 min. The coating was washed/leached to remove any non bonded residual heparin by placing the film in 250 ml of water and place in a mechanical shaker overnight. According to the above described whole blood test the surface showed no blood coagulation.

### Example 32

Coating Variation

A polyurethane film was primed and coated with the formula 5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid which was mixed 1:1 with a Salicylic Acid solution containing 0.08 g SA and 0.1118 g succinic semialdehyde in 10 ml of water (previously mixed) by using a stainless steel wire drawdown rod#10. This layered system was cured for 30 min at 120 C and is lubricious. According to the above described whole blood test the surface showed blood coagulation.

### Example 33

Anticoagulation Coating with Non-Leaching Heparin

A polyurethane film was primed and coated with the formula 5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by using a stainless steel wire drawdown rod#10. This layered system was cured for 30 min at 120 C. A 3% heparin formulation in water with 22% lactic acid and 2.8% Carbodilite E-03A was used as topcoat and applied with a stainless steel wire drawdown rod #10 and cured at 120 C for 30 min. The coating was washed/leached to remove any non bonded residual heparin by placing the film in 250 ml of water and place in a mechanical shaker overnight. According to the above-described whole blood test the surface showed no clot and a dark purple staining with TB.

### Example 34

Anticoagulation Coating with Non-Leaching Heparin

Three different polyurethane tubes (white, yellow, clear) were primed and coated with the formula 5 g of Chitosan mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by dipcoating. This layered system was cured for 60 min at 100 C. A 3% heparin formulation in water with 9% lactic acid, 0.7% acrylic acid, 20% IPA, 5% NMP, 5% THF and 0.1% FC-170 was used as topcoat and dipcoated, cured at 100C for 60 min then at RT overnight and additionally cured for 1 hrs 100 C. The coating was washed/leached to remove any non-bonded residual heparin by placing the film in 250 ml of water and place in a mechanical shaker overnight. According to the above-described whole blood test the surface showed no clot and an even-dark purple staining with TB.

### Example 35

Anticoagulation Coating with Non-Leaching Heparin

Polyurethane tubing (white, yellow, clear) were primed and coated with the formula 5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by dipcoating. This layered system was cured for 60 min at 100 C. A 3% heparin formulation in water with 22% lactic acid and 2.8% Carbodilite E-03A was used as topcoat and applied by dipcoating, cured at 100 C for 60 min then at RT overnight and additionally 1 hrs 100 C. The coating was washed/leached to remove any non bonded residual heparin by placing the film in 250 ml of water and place in a mechanical shaker for 4 hours. According to the above-described whole blood test the surface showed no clot and a medium to light even purple staining with TB.

### Example 36

Anticoagulation Coating with Non-Leaching Heparin

Polyurethane tubing (white, yellow, clear) were primed and coated with the formula 5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by dipcoating. This layered system was cured for 60 min at 100 C. A 3% heparin sodium salt formulation in water with 22% lactic acid was used as topcoat and applied by dipcoating, cured at 100 C for 60 min then at RT overnight and additionally 1 hrs 100 C. The coating was washed/leached to remove any non bonded residual heparin by placing the film in 250 ml of water and place in a mechanical shaker for 4 hours. According to the above-described whole blood test the surface showed no clot and an even purple staining with TB.

### Example 37

1 g Chitosan and 3 g Semialdehyde was mixed into 20 g IPA, 66 g Water, 5 g Tetrahydrofuran 5 g N-Methylpyrrolidone, 0.1 g Acrylic Acid and 0.02 g FC-170. The formulation was used to coat a polyurethane film and was cured for 30 min at 120 C.

The formulation forms a stable network upon evaporation of the carrier solvents and becomes lubricious upon contact with saline solution. By the touch the coating appears to be less lubricious than the coating with lactic acid instead of semialdehyde.

### Example 38

1 g Chitosan and 3 g Lactic Acid was mixed into 20 g IPA, 66 g Water, 5 g Tetrahydrofuran 5 g N-Methylpyrrolidone, 0.1 g Glutaraldehyde and 0.02 g FC-170. The formulation was used to coat a polyurethane film and was cured for 30 min at 120 C.

The formulation forms a stable network upon evaporation of the carrier solvents and becomes lubricious upon contact with saline solution. By the touch the coating appears very lubricious but slightly less than the coating with acrylic acid instead of glutaraldehyde.

### Example 39

5 g Chitosan are dissolved in 100 g Isopropanol, 25 g NMP, 25 g THF and 329.4 g of deionized water with 15 g of Lactic Acid, 0.5% Acrylic Acid and 0.1 g FC-170. A primer was applied first to prime a stainless steel substrate and than coated with the formulation as topcoat by dipping. The coating is lubricious, has good adhesion and shows good uniformity when stained with a water-soluble food color(FD&D Blue).

### Example 40

Anticoagulation Coating with Non-Leaching Heparin on Stents

Stents were primed and coated with the formula 5 g of Chitosan mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by spraying. This layered system was cured for 30 min at 120 C. A 3% heparin solution in water with 22% lactic acid was used as topcoat and applied by spraying and cured at 120 C for 30 min. The coatings on the stents were washed/leached to remove any non bonded residual heparin by placing the stents in 2 ml PBS and leach/washed in a mechanical shaker overnight. According to the above-described whole blood test and prothrombin test the wash solution was tested regarding prothrombin time. The control as well the leaching solutions had prothrombin times around 10 to 13 seconds. The subsequent test was conducted with the coated stents with plasma and whole blood. Each stent was incubated for 1 hr at RT in 2 ml citrated human whole blood. Then 100 ul of whole blood was mixed with 100 ul Thromboplastin and the time for clot formation recorded. In addition, 0.50 ml of this whole blood was centrifuged and the plasma was tested for prothrombin time and clotting. The control had a PT time of 16.9 sec, with whole blood and plasma clotting was observed, but non of the stents showed whole blood- or plasma clotting with PT times over 100 sec. The stent surface was also showing a purple stain when dipped in Toluidine Blue solution indicating the presences of heparin, however bonded (non-leaching).

### Example 41

Carrier for Drug Elution

1 g Chitosan and 2 g Aspirin was mixed into 20 g IPA, 67 g Water, 5 g Tetrahydrofuran 5 g N-Methylpyrrolidone and 0.02 g FC-170. The formulation was used to coat a polyurethane film and cured for 30 min at 120 C. By ultrasonic extraction for 30 min, 60 min and 90 min the corresponding amounts of 67%, 83% and 100% salicylic acid (hydrolyzed aspirin) were recovered by HPLC analysis.

In a second test the coated film was subject to RT leaching in PBS. After 24 hrs 40%, after 60 hrs 51% and after 96 hrs 76% of salicylic acid was recovered.

### Example 42

1 g Chitosan and 3 g salicylic acid mixed into 20 g IPA, 65.88 Water, 5 g Tetrahydrofuran 5 g N-Methylpyrrolidone, 0.1 g acrylic acid and 0.02 g FC-170. The formulation was used to coat a polyurethane film and cured for 30 min at 120 C. The coating exhibits similar lubricity by the touch when wet.

The coating was than topcoated with a 1% heparin solution with 3% lactic acid in water, cured at 120 C for 30 min and leached in PBS at RT over night. With the blood test described above it showed no blood clot formation upon contact of whole human blood.

### Example 43

No Red Blood Cell Hemolysis

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid. The formulation was used to coat 18 mm glass cover slips. 2 ml of citrated human blood was incubated for 1, 3 and 7 hrs at 37 C in the presence of the coated cover slips. 0.5 ml was centrifuged and the absorbance of a 1:20 dilution in PBS was recorded for the referenced incubation times. For up to 7 hrs the blood cells did not experience hemolysis evidenced by the practically stable visible light absorption at 600 nm ranging from 0.125 to 0.15 relative intensity.

### Example 44

Retardation of Cell Proliferation

5 g of Chitosan were mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid.

With the coating formulation 18 mm glass cover slips were coated. Cells were seeded in a 12 well tissue culture plate containing blank (control) and coated glass slips. After 3 and 5 days of growth the cover slips were rinsed in PBS and placed into a new 12 well dish containing 0.5 ml fresh media. 25 ul of MTS tetrazolium reagent was added and samples incubated for 3 hrs at 37 C. Cell proliferation was identified by the relative visual light absorption at 492 nm of the reagent stained cells. In the presence of Murine L929 cells and human aortic muscle cells with a blank, a control and the F200 coated samples. F200 coated samples had a 67% cell proliferation retatdation towards L929 cells versus the control after 3 days of growth. Smooth muscle cells were retartdeed in their proliferation by 76% after 3 days of growth and 95% after 5 days indicating a near zero cell mitosis.

### Example 45

Platelet Adhesion Prevention

1 g Chitosan and 1.5 g and 3 g acetyl salicylic acid respectively was mixed into 20 g IPA, 67 g Water, 5 g Tetrahydrofuran 5 g N-Methylpyrrolidone, 0.1 g acrylic acid and 0.02 g FC-170. The formulation was used to coat a glass cover slips and cured for 30 min at 120 C. The slides were tested according to the “Assays of Measuring Platelet Adhesion” as mentioned above. The incorporation of acetyl salicylic acid in place of lactic acid resulted in significant reduction in the adherence of human platelets to the coated glass surfaces.

### Example 46

Lubricious Coating for Various Substrates

1 g Chitosan and 3 g lactic acid was mixed into 20 g IPA, 67 g Water, 5 g Tetrahydrofuran 5 g N-Methylpyrrolidone, 0.1 g acrylic acid and 0.02 g FC-170. The formulation was used with and without primers to coat various substrates. Silicone membranes, polypropylene tubes and stainless steal plates showed good adhesion and significant slipperiness upon contact with water.

### Example 47

Contact Angle

Polyurethane film was coated with the formulation of 1 g Chitosan and 3 g lactic acid in 20 g IPA, 67 g Water, 5 g Tetrahydrofuran 5 g N-Methylpyrrolidone, 0.1 g acrylic acid and 0.02 g FC-170. The contact angles were determined at various times and temperatures of curing condition with subsequent wetting times of 0, 30 and 60 seconds. The uncoated film had a contact angle of 90 degrees for all wetting times. Low contact angles were found for curing temperatures of from 80 C to 140 and 20 to 60 min curing time ranging from 27 degrees to 80 degrees.

### Example 48

Antimicrobial Surface Testing

A polyurethane film was coated with the formula 5 g of Chitosan mixed with 15 g of Lactic Acid into 100 g of Isopropyl alcohol, 25 g Tetrahydrofuran, 25 g N-methylpyrrolidone, 0.1 g FC-170, 329.40 g water and 0.5 g acrylic acid by using a stainless steel wire drawdown rod#10. The coating was cured for 30 min at 120 C. The antimicrobial surface activity of the coated polyurethane film was tested according to the method described above. Against Escherichia coli, ATCC# 25922, the surface stayed active after 1 and 2 weeks of leaching. No colonization on the surface was observed after 2 weeks of leaching. Against Pseudomonas aeruginosa, ATCC# 27853 the surface stayed active for at least 1 week. No colonization on the surface was observed after 1 week of leaching.

### Example 49

A 3% heparin sodium salt formulation in water with 15% lactic acid and 0.3% acrylic acid used to coat a polyurethane film applied by dipcoating, cured at 100 C for 60 min. The coating was washed/leached to remove any non bonded residual heparin by placing the film in 250 ml of water and place in a mechanical shaker for 4 hours.

The coating is adhering well to the substrate. According to the above-described whole blood test the surface showed no clot and an even purple staining with Toluidine Blue.

1. A network composition formed by contacting:
a multifunctional monomer component consisting of a first monomer and a second monomer,
wherein the first monomer has one set of functional groups selected from the list consisting of hydroxyl/aldehyde; aldehyde/carboxylic acid; hydroxyl/carboxylic acid; aldehyde/amine; carboxylic acid/amine; amine/amine; carboxylic acid/carboxylic acid; hydroxyl/amine; hydroxyl/hydroxyl; aldehyde/aldehyde and combinations thereof, and
wherein the second monomer has at least one set of functional group selected from the list consisting of hydroxyl/carboxylic acid; carboxylic acid/carboxylic acid; hydroxyl/aldehyde; aldehyde/carboxylic acid; aldehyde/aldehyde; hydroxyl/hydroxyl; carboxylic acid/vinyl; amine/carboxylic acid; amine/amine; hydroxyl/vinyl/carboxylic acid; hydroxyl/olefin/carboxylic acid; olefin/carboxylic acid; carboxylic anhydride; carboxylic acid anhydride/hydroxyl; carboxylic acid anhydride/aldehyde; carboxylic acid anhydride/olefin; carboxylic acid anhydride/vinyl; carboxylic acid anhydride/amine; hydroxyl/olefin; hydroxyl/amine; aldehyde/olefin; aldehyde/vinyl; aldehyde/amine; aziridine; aziridine derivatives; epoxides; blocked isocyanates; colloidal silica; colloidal alumina; and combinations thereof,
wherein the ratio of the first monomer to the second monomer is from about 5:1 to about 50:1, or wherein the second monomer is absent; and
a saccharide component containing functional groups which can associate with the first and second monomers, wherein the weight ratio of the saccharide component to the monomer component ranges from about 1:50 to about 10:1;
wherein the monomer component and the saccharide component are contacted in the presence of a solvent composition; and
wherein upon evaporation of the solvent composition the network composition forms.

2. The network composition according to claim 1 wherein upon evaporation of the solvent composition the film adheres to a substrate.

3. The network composition according to claim 2 wherein the coefficient of friction of the substrate is reduced by at least about 85%.

4. The network composition according to claim 2 wherein the substrate is a medical device.

5. The network composition according to claim 1 wherein the ratio of the first monomer to the second monomer is from about 20:1 to about 30:1.

6. The network composition according to claim 1 wherein the weight ratio of the saccharide component to the monomer component is from about 1:10 to about 2:1.

7. The network composition according to claim 1 wherein the first monomer and the second monomer each individually comprise from about 2 to about 24 carbon atoms.

8. The network composition according to claim 1 wherein the saccharide component comprises polysaccharides, oligosaccharides, trisaccharides, disaccharides, monosaccharides, or derivatives thereof or combinations thereof.

9. The network composition according to claim 8 wherein the saccharide component comprises polyols, cellulosics, chitosans, heparin, starch, sugar, homopolysaccharides, heteropolysaccharides, glucosamines, or derivatives thereof, or combinations thereof.

10. The network composition according to claim 9 wherein the chitosans and chitosan derivatives are chosen from the group consisting of chitin, deacetylated chitin, N-carboxymethyl chitosan, O-carboxymethyl chitosan, N,O-carboxymethyl chitosan, carboxypropyl chitosan, carboxybutyl chitosan, hydrolized chitosan, chitosan adipate, chitosan ascorbate, chitosan formate, chitosan glycolate, polyquaternium-29, chitosan PCA (pyrrolidone carboxylic acid salt of chitosan), myristoyl/PCA chitin, chitosan lactate, chitosan lauroyl glycinate, chitosan salicylate, chitosan succinimide, galactosylated chitosan, hydroxyethyl chitosan, hydroxypropyl chitosan, and amino derivatives thereof, aldehyde derivatives thereof, carboxylic acid derivatives thereof, and combinations thereof.

11. The network composition according to claim 9 wherein the cellulosics are chosen from the group consisting of cellulose, polyquaternium-4, polyquaternium-10, polyquaternium-4/hydroxypropyl starch copolymer, polyquaternium-24, cellulose acetate, cellulose acetate butyrate, cellulose acetate propionate, cellulose acetate propinate carboxylate, cellulose gum, cellulose succinate, carboxy cellulose, aminocellulose, aminocellulose tosylates, and amino derivatives thereof, aldehyde derivatives thereof, carboxylic acid derivatives thereof, and combinations thereof.

12. The network composition according to claim 8 wherein the polysaccharides and polysaccharide derivatives are chosen from the group consisting of corn starch, hydrolyzed wheat protein, hydrolyzed wheat protein/PVP crosspolymer, glycogen, gelatin, inulin, pectin, heparin salts, hyaluronan, carregannan, algennan, algenic acid, alginate, gum arabic, locust bean gum, agar-agar, carrageenans, guar gum, xanthan gum, aloe barbadesis polysaccharides, arbutin, glucosic acid, glucodides, their amino derivatives, aldehyde derivatives, carboxylic acid derivatives and combinations thereof.

13. The network composition according to claim 9 wherein the sugar is glucose, fructose, mannose, galactose, algae oligosaccharides, their amino derivatives, aldehyde derivatives, carboxylic acid derivatives, d-(alpha or beta)glycosamine, d-(alpha or beta)galactoseamine and alkyl derivatives of these amino sugars.

14. The network composition according to claim 8 wherein the saccharide component comprises functional groups selected from the list consisting of hydroxyl; aldehyde; carboxylic acid; carboxyalkyl acid; amine; alkyl-amine; vinyl saccharides, saccharides containing olefinic side chains, saccharide isocyanates, —SH, —S-Alkyl, —SO4-, —SO3-, sulfonamides, SNH-alkyl; and combinations thereof.

15. The network composition of claim 1 wherein the first monomer is selected from the group consisting of an alcohol; an aldehyde; an glutaraldehyde; lactic acid; salicylic acid; p-hydroxybenzoic acid; citric acid; glycerin acid; alanine; glutamic acid; a primary amine; a carboxylic acid; dicarboxylic acid anhydride; a hydroxydicarboxylic acid; an alpha-amino acid; a beta-amino acid; a gamma-amino acid; an omega-amino acid; an alpha-hydroxy carboxylic acid; a beta-hydroxy carboxylic acid; a gamma-hydroxy carboxylic acid, an omega-hydroxy carboxylic acid; an alpha-hydroxy aldehyde; a beta-hydroxy aldehyde; a gamma-hydroxy aldehyde; an omega-hydroxy aldehyde; an alpha-aldehyde carboxylic acid; a beta-aldehyde carboxylic acid; a gamma-aldehyde carboxylic acid; an omega-aldehyde carboxylic acid; a diamine; and a hydroxy amine.

16. The network composition of claim 1 wherein the second monomer is selected from the group consisting of acrylic acid, an alcohol; an aldehyde; an glutaraldehyde; aspartamic acid; aspartame; lactic acid; salicylic acid; p-hydroxybenzoic acid; maleic acid; citric acid; sorbic acid; glycerin acid; alanine; glutamic acid; a primary amine; a carboxylic acid; a dicarboxylic acid anhydride; a hydroxydicarboxylic acid; an alpha-amino acid; a beta-amino acid; a gamma-amino acid; an omega-amino acid; an alpha-hydroxy carboxylic acid; a beta-hydroxy carboxylic acid; a gamma-hydroxy carboxylic acid, an omega-hydroxy carboxylic acid; an alpha-hydroxy aldehyde; a beta-hydroxy aldehyde; a gamma-hydroxy aldehyde; an omega-hydroxy aldehyde; an alpha-aldehyde carboxylic acid; a beta-aldehyde carboxylic acid; a gamma-aldehyde carboxylic acid; an omega-aldehyde carboxylic acid; a diamine; a hydroxy amine; an alpha-olefinic carboxylic acid; a beta-olefinic carboxylic acid; a gamma-olefinic carboxylic acid; an omega olefinic carboxylic acid; an alkylated acrylic acid; a hydroxyalkylated acrylic acid; an amino acrylic acid; an aminoalkylated acrylic acid; an alpha dimethylacrylic acid; a beta dimethylacrylic acid; a hydroxyacrylic acid, a semialdehyde; ginipin; hydroxyethylmethylacrylate (HEMA); hydroxypropyl methylacrylate (HPMA); colloidal silica; colloidal alumina; an epoxide; a melamine, an aziridine; a carbodiimide; a blocked di-isocyanate; a blocked multiisocyanate; a blocked di-thioisocyanate; and a blocked multithioisocyanate.

17. The network composition according to claim 1 wherein the solvent composition comprises water, alcohols, alkylketones, arylalkylketones, ketoalcohols, cyclic ketones, heterocyclic ketones, ethers, cyclic ethers, esters and combinations thereof

18. The network composition according to claim 17 wherein the solvent composition comprises methanol, ethanol, propanol, isopropanol, butanol, methyl ethyl ketone, tetrahydrofuran, acetone, diacetone alcohol, N-methylpyrrolidone, dimethylsulfoxide, (DMSO), dimethylformamide (DMF) and combinations thereof.

19. The network composition according to claim 1 further comprising a film-improving ingredient, a biologically active material or a combination of both.

20. The network composition according to claim 19 wherein the film-improving ingredient is selected from the group consisting of a surfactant, a wetting agent, a plasticizer, a humectant, a viscosity modifier, a defoamer, an emulsifier, a dye, a pigment, a colorant, a UV absorber, a radical scavenger, an antioxidant, an anti-corrosion agent, a carbon dioxide releaser, an optical brightener, a fluorescer, a bleach, a bleach activator, a bleach catalyst, a non-activated enzyme, an enzyme stabilizing system, a chelant, a coating aid, a metal catalyst, a metal oxide catalyst, an organometallic catalyst, a film forming promoter, a hardener, a linking accelerator, a flow agent, a leveling agent, a lubricant, a matte particle, a rheological modifier, a thickener, an electrolyte, a conductive or non-conductive metal oxide particle, a colloidal antimicrobial metal oxide, a magnetic particle, an anti-static agent, a pH control agent, a perfume, a preservative, a biocide, a pesticide, an anti-fouling agent, an algicide, a bactericide, a germicide, a disinfectant, a fungicide, a bio-effecting agent, a vitamin or combinations thereof.

21. The network composition according to claim 19 wherein said biologically active material is an antithrombotic agent, a biostatic agent, a cytostatic agent, a radiation emitter, a pharmaceutical, a biomolecule, an anti-inflammatory agent, an immunosuppressant, an antibiotic, an antiseptic, or combinations thereof.

22. The network composition according to claim 21 wherein said biologically active material is linked by chemical functional group interaction to the composition or is physically embedded in the composition.

23. The network composition according to claim 1 wherein the saccharide component comprises heparin, or derivatives thereof, or combinations thereof.

24. A method of making a network composition comprising:
contacting a multifunctional monomer component and a saccharide component in the presence of a solvent composition to form an reaction solution, and
evaporating the solvent composition to form the network composition, wherein the multifunctional monomer component comprises a first monomer and a second monomer,
wherein the first monomer has one set of functional groups selected from the list consisting of hydroxyl/aldehyde; aldehyde/carboxylic acid; hydroxyl/carboxylic acid; aldehyde/amine; carboxylic acid/amine; amine/amine; carboxylic acid/carboxylic acid; hydroxyl/amine; hydroxyl/hydroxyl; aldehyde/aldehyde and combinations thereof, and
wherein the second monomer has at least one set of functional group selected from the list consisting of hydroxyl/carboxylic acid; carboxylic acid/carboxylic acid; hydroxyl/aldehyde; aldehyde/carboxylic acid; aldehyde/aldehyde; hydroxyl/hydroxyl; carboxylic acid/vinyl; amine/carboxylic acid; amine/amine; hydroxyl/vinyl/carboxylic acid; hydroxyl/olefin/carboxylic acid; olefin/carboxylic acid; carboxylic anhydride; carboxylic acid anhydride/hydroxyl; carboxylic acid anhydride/aldehyde; carboxylic acid anhydride/olefin; carboxylic acid anhydride/vinyl; carboxylic acid anhydride/amine; hydroxyl/olefin; hydroxyl/amine; aldehyde/olefin; aldehyde/vinyl; aldehyde/amine; aziridine; aziridine derivatives; epoxides; blocked isocyanates; colloidal silica; colloidal alumina; and combinations thereof,
wherein the ratio of the first monomer to the second monomer is from about 5:1 to about 100:1, or wherein the second monomer is absent; and
wherein the saccharide component contains functional groups which can associate with the first and second monomers, wherein the weight ratio of the saccharide component to the monomer component ranges from about 1:50 to about 10:1.

25. The method of claim 24 wherein the saccharide component makes up about 0.01 wt. % to about 20 wt. % of the reaction solution.

26. The method of claim 25 wherein the saccharide component makes up about 0.1 wt. % to about 10 wt. % of the reaction solution.

27. The method of claim 24 wherein the multifunctional monomer component makes up about 0.001 wt. % to about 30 wt. % of the reaction solution.

28. The method of claim 27 wherein the multifunctional monomer component makes up about 0.01 wt. % to about 15 wt. % of the reaction solution.

29. The method of claim 24 wherein the solvent composition makes up about 99.99% wt. to about 50% wt. % of the reaction solution.

30. The method of claim 24 wherein the solvent composition comprises water, alcohols, alkylketones, arylalkylketones, ketoalcohols, cyclic ketones, heterocyclic ketones, ethers, cyclic ethers, esters and combinations thereof.

31. The network composition according to claim 30 wherein the solvent composition comprises methanol, ethanol, propanol, isopropanol, butanol, methyl ethyl ketone, tetrahydrofuran, acetone, diacetone alcohol, N-methylpyrrolidone, dimethylsulfoxide, (DMSO), dimethylformamide (DMF) and combinations thereof.

32. The method of claim 24 further comprising contacting with a film-improving ingredient, a biologically active material, or combinations thereof.

33. The method of claim 24 wherein the film-improving ingredient and/or the biologically active material makes up about 0.001% wt. to about 3% wt. %

34. An object which comprises the network composition of claim 1, wherein the composition adheres to the object.

35. The object of claim 34 wherein the object is made of metal, stainless steel, Nitinol®, plastics, polymers, glass, ceramics, cellulose fibers, synthetic fibers, textiles and alike.

36. The object according to claim 34 wherein said object is a medical device.

37. The object according to claim 36 wherein said medical device is a non-expendable or expendable stent, catheter, guide wire, shunt, screw, pin, prosthesis, plate, film, sponge, suture, medical tubing, cannula, balloon, needle, marker, stylet, surgical rod, guidewire tubes, coiled guiding tubes, coiled catheters, electrodal coils, blades, fibers, wound dressing fibers, band aids, suture threads, ocular lens delivery device, ocular lenses, ocular catheters, dialysis catheters, wound drain, or bone implants.

38. The object according to claim 34 wherein the network composition is applied to the object by dipping, spraying, flooding, foaming, roll-coating, brushing, electrolytic depositing, electrostatic spraying, electroplating, vacuum treatment, pressure treatment or combinations thereof.

39. A network composition comprising a plurality of associated saccharide chains
wherein a chain comprises at least one saccharide component; and at least one first monomer, at least one second monomer, or combinations of both;
wherein the first monomer is linked to the saccharide component by ester linkages; ether linkages; amide linkages; ketone linkages; or combinations thereof;
wherein the second monomer is linked to the saccharide component by ester linkages; ether linkages; amide linkages; ketone linkages; urea linkages; carbamate linkages, aluminum oxide linkages; siloxane linkages; or combinations thereof;
wherein first monomers are linked to each other by ester linkages; ether linkages; amide linkages; ketone linkages; or combinations thereof;
wherein the first monomer is linked to the second monomer by ester linkages; ether linkages; amide linkages; ketone linkages; urea linkages; carbamate linkages; aluminum oxide linkages; siloxane linkages or combinations thereof;
wherein the second monomers are linked to each other by ester linkages; ether linkages; amide linkages; ketone linkages; polyvinyl linkages, polyolefin linkages, urea linkages; carbamate linkages, aluminum oxide linkages, siloxane linkages or combinations thereof; and
wherein the saccharide components are linked to each other by ester linkages; ether linkages; amide linkages; ketone linkages; or combinations thereof;
wherein the network is capable of adhering to a substrate.

40. The lubricious network composition of claim 39 wherein the first monomer and second monomer each individually comprise about two to about 25 carbon atoms.

41. The network composition according to claim 39 wherein the saccharide component comprises polysaccharides, oligosaccharides, trisaccharides, disaccharides, monosaccharides, or derivatives thereof or combinations thereof.

42. The network composition according to claim 41 wherein the saccharide component comprises polyols, cellulosics, chitosans, heparin, starch, homopolysaccharides, heteropolysaccharides, glucosamines, or derivatives thereof, or combinations thereof.

43. The network composition according to claim 42 wherein the chitosans and chitosan derivatives are chosen from the group consisting of chitin, deacetylated chitin, N-carboxymethyl chitosan, O-carboxymethyl chitosan, N, O-carboxymethyl chitosan, carboxypropyl chitosan, carboxybutyl chitosan, hydrolized chitosan, chitosan adipate, chitosan ascorbate, chitosan formate, chitosan glycolate, polyquaternium-29, chitosan PCA (pyrrolidone carboxylic acid salt of chitosan), myristoyl/PCA chitin, chitosan lactate, chitosan lauroyl glycinate, chitosan salicylate, chitosan succinimide, galactosylated chitosan, hydroxyethyl chitosan, hydroxypropyl chitosan, and amino derivatives thereof, aldehyde derivatives thereof, carboxylic acid derivatives thereof, and combinations thereof.

44. The network composition according to claim 42 wherein the cellulosics are chosen from the group consisting of cellulose, polyquaternium-4, polyquaternium-10, polyquaternium-4/hydroxypropyl starch copolymer, polyquaternium-24, cellulose acetate, cellulose acetate butyrate, cellulose acetate propionate, cellulose acetate propinate carboxylate, cellulose gum, cellulose succinate, carboxy cellulose, aminocellulose, aminocellulose tosylates, and amino derivatives thereof, aldehyde derivatives thereof, carboxylic acid derivatives thereof, and combinations thereof.

45. The network composition according to claim 41 wherein the polysaccharides and polysaccharide derivatives are chosen from the group consisting of hydrolyzed wheat protein, hydrolyzed wheat protein/PVP crosspolymer, glycogen, gelatin, inulin, pectin, heparin salts, hyaluronan, carregannan, algennan, algenic acid, alginate, gum arabic, locust bean gum, agar-agar, carrageenans, guar gum, xanthan gum, aloe barbadesis polysaccharides, arbutin, glucosic acid, glucodides, their amino derivatives, aldehyde derivatives, carboxylic acid derivatives and combinations thereof.

46. The network composition according to claim 41 wherein the saccharide component is glucose, fructose, mannose, galactose, algae oligosaccharides, their amino derivatives, aldehyde derivatives, carboxylic acid derivatives, d-(alpha or beta)glycosamine, d-(alpha or beta)galactoseamine and alkyl derivatives of these amino sugars and combinations thereof.

47. The network composition according to claim 41 wherein the saccharide component comprises heparin, or derivatives thereof, or combinations thereof.
